(19) United States
(12) Patent Application Publication
(10) Pub. No.: US 2018/0218789 A1
(43) Pub. Date: Aug. 2, 2018

(54) METHODS AND SYSTEMS FOR SEQUENCING-BASED VARIANT DETECTION

(71) Applicant: Farsight Genome Systems, Inc., Sunnyvale, CA (US)

(72) Inventors: Glenda G. Anderson, San Jose, CA (US); Charlie C. Kim, Santa Clara, CA (US)

(21) Appl. No.: 15/862,068

(22) Filed: Jan. 4, 2018

Related U.S. Application Data
(63) Continuation of application No. PCT/US2016/041288, filed on Jul. 7, 2016.
(60) Provisional application No. 62/189,555, filed on Jul. 7, 2015.

(51) Int. Cl. G16H 50/20; C12Q 1/6883; C12Q 1/6869; G16H 10/40; C12Q 1/6848

(52) U.S. Cl. CPC G16H 50/20; C12Q 1/6883; C12Q 1/6869; C12Q 2600/166; C12Q 1/6848; C12Q 2600/106; C12Q 2600/156; G16H 10/40

(57) ABSTRACT

Provided herein are methods and systems for detecting genetic variants from sequencing data. The methods and systems provided herein can be useful for identifying the presence or absence of clinically actionable variants from a sequencing data set and reporting the clinically actionable variants to a user of the methods and systems.

### CROSS REFERENCE

This application is a continuation application of International Patent Application No. PCT/US2016/041288, filed on Jul. 7, 2016, which application claims the benefit of U.S. Provisional Application No. 62/189,555, filed Jul. 7, 2015, which application is incorporated herein by reference in its entirety.

### BACKGROUND OF THE INVENTION

Sequencing is rapidly becoming an important tool in the diagnostic workup of solid tumors. Of the more than 700 oncology drugs in the clinical development pipeline, 73% are expected to require a biomarker. The ability to distinguish the true presence and true absence of clinically actionable variants may find utility in the personalized medicine field. However, current variant calling algorithms and methods are not able to positively identify the absence of a variant. This limitation has unfavorable consequences for laboratory validation methods that require both true positive and true negative calls to quantify test sensitivity and specificity. This limitation has unfavorable impact on clinical decision-making, most notably with variants whose absence guides the choice of treatment. Improved software systems are needed to manage the complexity of multiple-marker testing.

### SUMMARY OF THE INVENTION

In one aspect, a method is provided for detecting the presence or absence of a genetic variant, comprising: a) receiving a data input comprising sequencing data generated from a nucleic acid sample from a subject; b) determining a presence or absence of the genetic variant from the sequencing data, wherein the determining comprises assigning a quality score to a genomic region comprising the genetic variant, wherein the assigning is performed by a computer processor; c) classifying the genetic variant based on the quality score to generate a classified genetic variant, and d) outputting a result based on the classifying, thereby identifying the classified genetic variant. In some cases, the classifying further comprises classifying the genetic variant as present if the genetic variant is determined to be present and the quality score for the genomic region comprising the genetic variant is greater than a predetermined threshold. In some cases, the classifying further comprises classifying the genetic variant as absent if the genetic variant is determined to be absent and the quality score for the genomic region comprising the genetic variant is greater than a predetermined threshold. In some cases, the classifying further comprises classifying the genetic variant as indeterminate if the quality score for the genomic region comprising the genetic variant is less than a predetermined threshold. In some cases, the outputting a result comprises generating a report, wherein the report identifies the classified genetic variant. In some cases, the method further comprises mapping the sequencing data to a reference sequence. In some cases, the reference sequence is a consensus reference sequence. In some cases, the reference sequence is derived empirically from tumor sequencing data. In some cases, the predetermined threshold comprises a depth of coverage of the genomic region comprising the genetic variant. In some cases, the depth of coverage is at least 10×. In some cases, the depth of coverage is at least 20×. In some cases, the depth of coverage is at least 30×. In some cases, the depth of coverage is at least 50×. In some cases, the depth of coverage is at least 100×. In some cases, the predetermined threshold comprises a confidence score. In some cases, the confidence score is at least 95%. In some cases, the confidence score is at least 99%. In some cases, the genetic variant comprises a clinically actionable variant. In some cases, the identifying the classified genetic variant further indicates a treatment for the subject based on the classified genetic variant. In some cases, the subject is suffering from a disease. In some cases, the disease is cancer. In some cases, the subject is administered a treatment based on the result. In some cases, the clinically actionable variant is in a gene that alters a response of the subject to a therapy. In some cases, the gene is a cancer gene. In some cases, a presence of a clinically actionable variant indicates the subject is a candidate for a specific therapy. In some cases, an absence of a clinically actionable variant indicates the subject is not a candidate for a specific therapy. In some cases, the nucleic acid sample is derived from blood or saliva. In some cases, the nucleic acid sample is derived from a solid tumor. In some cases, the nucleic acid sample is genomic DNA. In some cases, the genomic DNA is tumor DNA. In some cases, the nucleic acid sample is RNA. In some cases, the RNA is tumor RNA. In some cases, the nucleic acid sample is derived from circulating tumor cells. In some cases, the nucleic acid sample comprises cell-free nucleic acids. In some cases, the genetic variant is a gene amplification, an insertion, a deletion, a translocation or a single nucleotide polymorphism. In some cases, the sequencing data comprises target-enriched sequencing data. In some cases, the target-enriched sequencing data comprises whole exome sequencing data. In some cases, the sequencing data comprises whole genome sequencing data. In some cases, the classifying has a sensitivity of at least 99%. In some cases, the classifying has a specificity of at least 99%. In some cases, the genetic variant, when classified as present, has a mutant allele fraction of at least 5%. In some cases, the genetic variant, when classified as present, has a mutant allele fraction of at least 10%. In some cases, the classifying has a positive predictive value of at least 99%. In some cases, the quality score is based on at least one of a depth of coverage, a mapping quality, or a base call quality. In some cases, the quality score is empirically determined. In some cases, the method further comprises transmitting the result over a network. In some cases, the network is the Internet. In some cases, the method further comprises, prior to step a), sequencing the nucleic acid sample from the subject to generate the sequencing data. In some cases, the method further comprises requerying the sequencing data to determine a presence or an absence of one or more additional genetic variants, comprising assigning a quality score to each of one or more genomic regions comprising the one or more additional genetic variants, wherein the quality score is classified as sufficient if the quality score is greater than a predetermined threshold and wherein the quality score is classified as insufficient if the quality score is lower than a predetermined threshold. In some cases, the quality score is determined by a total read depth at a specific location of the genetic variant, a proportion of reads containing the genetic variant, the mean quality of non-variant base calls at the location of the genetic variant, and the difference in mean quality for variant base calls. In some cases, the quality score is determined by a machine learning algorithm. In some cases, the method is utilized as a clinical diagnostic.

In another aspect, a method is provided for modifying a sequencing protocol comprising: a) receiving a data input comprising sequencing data generated by the sequencing protocol; b) determining a presence or absence of a genetic variant from the sequencing data, wherein the determining comprises assigning a quality score to a genomic region comprising the genetic variant, wherein the assigning is performed by a computer processor; c) classifying the genetic variant based on the quality score to generate a classified genetic variant; d) outputting a result based on the classifying, thereby identifying the classified genetic variant. In some cases, the genetic variant is classified as present if the genetic variant is determined to be present and the quality score is greater than a predetermined threshold. In some cases, the genetic variant is classified as absent if the genetic variant is determined to be absent and the quality score is greater than a predetermined threshold. In some cases, a modification to the sequencing protocol is made if the quality score is lower than a predetermined threshold. In some cases, the outputting a result comprises generating a report, wherein the report identifies the classified genetic variant. In some cases, the method further comprises mapping the sequencing data to a reference sequence. In some cases, the reference sequence is a consensus reference sequence. In some cases, the reference sequence is derived empirically from tumor sequencing data. In some cases, the genetic variant is a clinically actionable variant. In some cases, the clinically actionable variant is in a gene that alters a response of the subject to a therapy. In some cases, the modification to the sequencing protocol comprises a modification to at least one of a probe, a primer, or a reaction condition. In some cases, the report is generated in real-time. In some cases, the predetermined threshold comprises a depth of coverage of the genomic region comprising the genetic variant. In some cases, the depth of coverage is at least 10×. In some cases, the depth of coverage is at least 20×. In some cases, the depth of coverage is at least 30×. In some cases, the depth of coverage is at least 50×. In some cases, the depth of coverage is at least 100×. In some cases, the predetermined threshold comprises a confidence score. In some cases, the confidence score is at least 95%. In some cases, the confidence score is at least 99%. In some cases, the quality score is based on at least one of a depth of coverage, a mapping quality, or a base call quality. In some cases, the quality score is empirically determined. In some cases, the sequencing data is generated from a nucleic acid. In some cases, the nucleic acid is genomic DNA. In some cases, the sequencing protocol comprises a target-enrichment protocol. In some cases, the target-enrichment protocol comprises at least one of target-specific primers and target-specific probes. In some cases, the modification comprises a modification to at least one of the target-specific primers and the target-specific probes. In some cases, the method further comprises receiving a second data input comprising second sequencing data generated from the modified sequencing protocol. In some cases, the modification to the sequencing protocol is determined by the result. In some cases, the method further comprises, prior to step a), sequencing the nucleic acid sample from the subject to generate the sequencing data. In some cases, the sequencing reaction is performed on a nucleic acid sample comprising the genetic variant. In some cases, the nucleic acid sample is isolated from a subject. In some cases, the subject is suffering from a disease. In some cases, the disease is cancer. In some cases, the method further comprises enriching for a nucleic acid sequence comprising the genetic variant prior to the sequencing reaction. In some cases, the enriching comprises hybridizing at least one target-specific probe to the nucleic acid sequence comprising the genetic variant. In some cases, the enriching comprises amplifying the nucleic acid sequence comprising the genetic variant. In some cases, the amplifying comprises hybridizing target-specific primers to the nucleic acid sample comprising the genetic variant. In some cases, the genetic variant is in an exon. In some cases, the method further comprises transmitting the result over a network. In some cases, the network is the Internet.

In another aspect, a system is provided for reporting the presence or absence of a genetic variant, comprising: a) at least one memory location configured to receive a data input comprising sequencing data generated from a nucleic acid sample from a subject; b) a computer processor operably coupled to the at least one memory location, wherein the computer processor is programmed to (i) determine a presence or absence of the genetic variant from the sequencing data, wherein the determining comprises assigning a quality score to a genomic region comprising the genetic variant to generate a classified genetic variant based on the quality score; and (ii) generate an output, wherein the output identifies the classified genetic variant. In some cases, the genetic variant is classified as present if the genetic variant is determined to be present and the quality score is greater than a predetermined threshold. In some cases, the genetic variant is classified as absent if the genetic variant is determined to be absent and the quality score is greater than a predetermined threshold. In some cases, the genetic variant is classified as indeterminate if the quality score is less than a predetermined threshold. In some cases, the output comprises a report identifying the classified genetic variant. In some cases, the report is delivered to a user interface for display. In some cases, the computer processor is programmed to map the sequencing data to a reference sequence. In some cases, the reference sequence is a consensus reference sequence. In some cases, the reference sequence is derived empirically from tumor sequencing data. In some cases, the genetic variant is a clinically actionable variant. In some cases, the clinically actionable variant is in a gene that alters a response of the subject to a therapy. In some cases, the report recommends a treatment based on the classified genetic variant. In some cases, the quality score is determined by at least one of depth of coverage, mapping quality, and base read quality. In some cases, the quality score is empirically determined. In some cases, the subject is suffering from a disease. In some cases, the disease is cancer. In some cases, the subject is predisposed to cancer. In some cases, the sequencing data comprises target-enriched sequencing data. In some cases, the target-enriched sequencing data comprises whole exome sequencing data. In some cases, the target-enriched sequencing data is generated from a target-enrichment sequencing protocol. In some cases, a modification to the target-enrichment sequencing protocol is made if the genetic variant is classified as indeterminate. In some cases, the at least one memory location is configured to receive a second data input comprising second sequencing data generated from the modification to the target-enrichment sequencing protocol. In some cases, the modification to the target-enrichment protocol comprises at least one modification to target-specific primers and target-specific probes. In some cases, the user interface is configured to enable a user to select a variant test panel. In some cases, the computer processor is programmed to determine a presence or absence of a genetic variant selected from the variant test panel. In some cases, the user interface is configured to enable a user to modify the variant test panel. In some cases, the user interface is configured to enable a user to add or remove at least one genetic variant from the variant test panel. In some cases, the user interface is operably coupled to at least one database. In some cases, the user interface receives a data input from the at least one database. In some cases, the variant test panel is updated in real-time based on the data input from the at least one database. In some cases, the variant test panel comprises at least one clinically actionable variant.

In yet another aspect, a system is provided comprising: a) a client component, wherein the client component comprises a user interface; b) a server component, wherein the server component comprises at least one memory location configured to receive a data input comprising sequencing data generated from a nucleic acid sample; c) the user interface operably coupled to the server component; and d) a computer processor operably coupled to the at least one memory location, wherein the computer processor is programmed to map the sequencing data to a reference sequence and assign a quality score to each of a plurality of genomic regions of interest of the mapped sequencing data. In some cases, (i) the user interface is programmed to enable a user to select at least one genetic variant and transmit the selection to the server component, wherein the genetic variant is located within at least one of the plurality of genomic regions of interest; (ii) the computer processor is programmed to return the quality score for at least one of the plurality of genomic regions of interest comprising the at least one genetic variant; and (iii) the computer processor is programmed to compare the quality score for at least one of the plurality of genomic regions of interest to a predetermined threshold, wherein the quality score is reported as sufficient if the quality score is greater than the predetermined threshold, and wherein the quality score is reported as insufficient if the quality score is lower than the predetermined threshold, and if the quality score is reported as sufficient, the computer processor is programmed to determine a presence or absence of each of the at least one genetic variant. In some cases, the genetic variant is classified as present if the genetic variant is determined to be present and the quality score is greater than the predetermined threshold. In some cases, the genetic variant is classified as absent if the genetic variant is determined to be absent and the quality score is greater than the predetermined threshold. In some cases, if the quality score is reported as insufficient, the computer processor is programmed to translate the at least one genetic variant into at least one chromosome location. In some cases, the server component transmits the at least one chromosome location to a third-party server component. In some cases, the quality score is determined by at least one of a depth of coverage, a mapping quality, and a base quality.

In another aspect, a method is provided comprising: (a) receiving a data input comprising sequencing data generated from a nucleic acid sample from a subject, wherein, prior to the receiving, the sequencing data has been analyzed and a presence or absence of one or more genetic variants has been identified, thereby generating an original analysis of the sequencing data; (b) assigning a quality score to each of one or more genomic regions of the sequencing data, the one or more genomic regions comprising at least one of the one or more genetic variants, wherein the assigning is performed by a computer processor; (c) evaluating the original analysis of the one or more genetic variants based on the quality scores, and (d) outputting a result based on the evaluating, wherein the evaluating further comprises identifying the original analysis for a genetic variant of the one or more genetic variants as accurate if the quality score for the genomic region comprising the genetic variant is greater than a predetermined threshold, and wherein the evaluating further comprises identifying the original analysis for a genetic variant of the one or more genetic variants as inaccurate if the quality score for the genomic region comprising the genetic variant is less than a predetermined threshold. In some cases, if the original analysis for a genetic variant is identified as inaccurate, the method further comprises recommending a modification to a sequencing protocol. In some cases, the predetermined threshold comprises a depth of coverage of the genomic region comprising the genetic variant. In some cases, the depth of coverage is at least 10×. In some cases, the depth of coverage is at least 20×. In some cases, the depth of coverage is at least 30×. In some cases, the depth of coverage is at least 50×. In some cases, the depth of coverage is at least 100×. In some cases, the predetermined threshold comprises a confidence score. In some cases, the confidence score is at least 95%. In some cases, the confidence score is at least 99%.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a computer system useful for performing the methods disclosed herein.

FIG. 2 depicts a non-limiting example of a report that can be generated by the methods and systems disclosed herein.

FIG. 3 depicts a non-limiting example of a report that can be generated by the methods and systems disclosed herein.

FIG. 4 depicts a non-limiting example of a report that can be generated by the methods and systems disclosed herein.

FIG. 5 depicts a non-limiting example of a report that can be generated by the methods and systems disclosed herein.

FIG. 6 depicts a non-limiting example of an exemplary study design described herein.

FIG. 7 depicts the identification of clinically-actionable variants using the methods and systems disclosed herein.

FIG. 8 depicts a confusion matrix illustrating the performance of the methods and systems disclosed herein.

FIG. 9 depicts box and whisker plots representing EGFR coverage analysis for 12 cohorts.

### DETAILED DESCRIPTION OF THE INVENTION

### Methods of the Disclosure

The disclosure herein provides methods for determining the presence or absence of genetic variants from sequencing data. The methods can comprise receiving a data input comprising sequencing data generated from a nucleic acid sample from a subject. The methods can further comprise determining a presence or absence of a genetic variant from the sequencing data. The determining step can comprise evaluating a data quality score for a genomic region comprising the genetic variant. The determining step can further comprise classifying the genetic variant based on the data quality score of the genomic region to generate a classified genetic variant. The methods can further comprise generating a report. The report can identify the classified genetic variant. In some cases, the genetic variant is classified as present if the genetic variant is determined to be present and the data quality score for the genomic region comprising the genetic variant is greater than a predetermined threshold. In other cases, the genetic variant is classified as absent if the genetic variant is determined to be absent and the data quality score for the genomic region comprising the genetic variant is greater than a predetermined threshold. In yet other cases, the genetic variant is classified as indeterminate if the data quality score for the genomic region comprising the genetic variant is less than a predetermined threshold.

The methods provided herein can be used for diagnosing a disease in a subject. The methods may further provide a treatment plan or recommendation based on the diagnosis. In some cases, the methods can be used to predict the responsiveness of a disease to a particular therapy. The methods disclosed herein utilize sequencing data generated from a nucleic acid sample and identify the presence or absence of genetic variants. The absence or presence of variants may indicate the responsiveness, or lack thereof, of a disease to a particular therapy. A report may be generated identifying the presence or absence of variants and a treatment recommendation based upon the presence or absence of the variants.

In some aspects, the methods herein provide for determining a presence or absence of genetic variants in a subject. A subject may submit a biological sample comprising nucleic acids. The subject can be healthy or can be suffering from a disease. In some cases, the subject may be predisposed to developing a disease. In particular cases, the subject is suffering from or is predisposed to developing cancer. In some cases, the subject is diagnosed with cancer. The subject may have a solid tumor and a sample can be taken (i.e., as a biopsy). In some cases, the methods disclosed herein can be ordered by a physician or health-care provider (e.g., as a genetic test). In some cases, the methods disclosed herein can be ordered by a clinical laboratory (e.g., a laboratory certified under the Clinical Laboratory Improvement Amendments (CLIA)). A biological sample can be tissue or cells taken from the subject (i.e. blood, cheek cells) or a substance produced by the subject (i.e. saliva, urine). In some cases, the biological sample is a biopsy of a tumor. In some cases, the sample is a formalin-fixed, paraffin-embedded (FFPE) tissue sample. The biological sample will generally comprise nucleic acid molecules. The nucleic acid molecules can be DNA or RNA, or any combination thereof. RNA can comprise mRNA, miRNA, piRNA, siRNA, tRNA, rRNA, sncRNA, snoRNA and the like. DNA can comprise cDNA, genomic DNA, mitochondrial DNA, exosomal DNA, viral DNA and the like. In particular cases, the DNA is genomic DNA. Nucleic acids can be isolated from biological cells or can be cell-free nucleic acids (i.e., circulating DNA). In particular examples, the DNA is tumor DNA. In other particular examples, the RNA is tumor RNA. In some cases, the DNA is fetal DNA.

The biological sample can be processed and analyzed by any number of steps to determine the presence or absence of a disease. The methods may comprise analyzing the biological sample for the presence or absence of biomarkers. The presence or absence of a biomarker can be indicative of a disease or of a predisposition for developing a disease. The presence or absence of a biomarker can indicate that a disease may be responsive to a particular therapy. In other cases, the presence or absence of a biomarker can indicate that a disease may be refractory to a particular therapy. A biomarker may be any gene or variant of a gene whose presence, mutation, deletion, substitution, copy number, or translation (i.e., to a protein) is an indicator of a disease state. In particular examples, a biomarker is a genetic variant. As used herein, the terms “variant”, “genetic variant” or “nucleotide variant” generally refer to a polymorphism within a nucleic acid molecule. A polymorphism may comprise one or more insertions, deletions, structural variants (e.g., translocations, copy number variations), variable length tandem repeats, single nucleotide mutations, or a combination thereof. In some cases, the genetic variant is a clinically actionable variant. A “clinically actionable variant” may be any genetic variant that has been identified as being relevant to the clinical setting. The clinically actionable variant can be in a coding region of a gene or can be in a non-coding region of the genome. The non-coding region of the genome can be a regulatory region of the gene. The clinically actionable variant can be in an exon of a gene or can be in an intron of a gene. A clinically actionable variant may alter the expression of the gene or may alter the function of the gene product (i.e., the function of the protein). A clinically actionable variant can regulate a gene involved in a disease. In particular examples, the clinically actionable variant alters the expression of or the function of a known cancer gene. In some cases, the clinically actionable variant alters the response of a protein to a therapy. For example, a clinically actionable variant may indicate that a protein is refractory to a specific therapy (e.g., a variant in an antigen such that an antibody therapy no longer recognizes the antigen). A clinically actionable variant can be in or regulate a target gene or can be in or regulate a gene other than the target gene. A gene other than the target gene can be a gene involved in drug metabolism, a gene involved in transport of drugs, genes associated with a favorable response to a particular drugs, DNA repair genes, genes that increase the severity of adverse events, and genes that alter the effectiveness of a drug.

Nucleic acid molecules can be processed and/or analyzed by any method known to one skilled in the art. In particular cases, the nucleic acid molecules are sequenced to generate sequencing data. Sequencing data can be generated by any known sequencing method (e.g., Illumina). Sequencing data may be generated from targeted sequencing methods or untargeted sequencing methods. The terms “target-specific”, “targeted,” and “specific” can be used interchangeably and generally refer to a subset of the genome that is a region of interest, or a subset of the genome that comprises specific genes or genomic regions. Targeted sequencing methods can allow one to selectively capture genomic regions of interest from a nucleic acid sample prior to sequencing. Targeted sequencing involves alternate methods of sample preparation that produce libraries that represent a desired subset of the genome or to enrich (“target enrichment”) the desired subset of the genome. Targeted sequencing can be, for example, whole exome sequencing. The terms “untargeted sequencing” or “non-targeted sequencing” can be used interchangeably and generally refer to a sequencing method that does not target or enrich a region of interest in a nucleic acid sample. The terms “untargeted sequence”, “non-targeted sequence,” or “non-specific sequence” generally refer to the nucleic acid sequences that are not in a region of interest or to sequence data that is generated by a sequencing method that does not target or enrich a region of interest in a nucleic acid sample. Untargeted sequencing can be, for example, whole genome sequencing. The terms “untargeted sequence”, “non-targeted sequence” or “non-specific sequence” can also refer to sequence that is outside of a region of interest. In some cases, sequencing data that is generated by a targeted sequencing method can comprise not only targeted sequences but also untargeted sequences.

The methods comprise receiving a data input comprising sequencing data generated from the nucleic acid sample from the subject. In some cases, the methods provide for receiving a data input comprising targeted sequencing data, untargeted sequencing data, or a combination of both. In some cases, the methods provide for receiving a data input comprising exonic sequencing data, non-exonic sequencing data, or a combination of both. Sequencing data can be received (i.e., by a computer) in any file format generated by the sequencing methods of the disclosure. The sequencing data may comprise additional information. For example, the sequencing data can comprise a nucleotide sequence and its corresponding quality scores (i.e., FASTQ file format).

The methods provide for analyzing the sequencing data. The sequencing data can be analyzed by one or more analysis methods. In some cases, the sequencing data can be mapped to a reference sequence. A reference sequence can be a canonical reference sequence. Canonical reference sequences can be found in, for example, a database (e.g., GENCODE, UCSC or EMBL). In other cases, the reference sequence may be derived empirically from sequencing data (e.g., from tumor sequencing data). In this example, the reference sequence can be created using read data from a large collection of similar cancer specimens that have been sequenced in uniform laboratory conditions (e.g., all lung samples from the Cancer Genome Atlas (TCGA) study). In some cases, each sample can be aligned to the canonical reference sequence before applying a sequence alignment algorithm (e.g., Feng-Doolittle, Barton-Strenberg, Gotoh, CLUSTALW, and the like). The root node of the resulting tree may represent the empirically-derived tumor reference sequence. In some cases, a multiple sequence alignment is performed from unaligned reads by profile Hidden Markov Model (HMM) training, using a combination of Baum-Welch, Viterbi or related approaches that use simulated annealing or consensus motif finding. In some cases, the computational complexity can be significantly reduced by subsetting the reads into gene or motif groups using a simple “best match” alignment algorithm. A multiple sequence alignment can then be performed within each subset to produce a gene-specific, or motif-specific, empirically-derived tumor reference sequence.

The methods further provide for determining a presence or absence of a genetic variant from the sequencing data. In some cases, the genetic variant can be a clinically actionable variant. Determining a presence or absence of a genetic variant can include assigning a quality score to a genomic region comprising the genetic variant and classifying the genetic variant based on the quality score to generate a classified genetic variant. The quality score can be determined by the read depth (or depth of coverage), the base quality, the mapping quality, or any combination thereof. In particular examples, the quality score is determined by the read depth of a genomic region of interest. A quality score can be assigned to a region of the sequencing data (a “regional” quality score) or can be assigned to the sequencing data as a whole. In some cases, the regional quality score may comprise a quality score of a specific variant. In particular cases, a regional quality score is assigned to a genomic region of interest. A “genomic region of interest” can be a region of the genome that is in the vicinity of the variant of interest. A genomic region of interest that is in the vicinity of the variant of interest can be within at most 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, 1 kb, 2 kb, 3 kb, 4 kb, 5 kb, 6 kb, 7 kb, 8 kb, 9 kb, 10 kb, 20 kb, 30 kb, 40 kb, 500 kb, 600 kb, 700 kb, 800 kb, 900 kb, 1000 kb or more of the variant of interest. The genomic region of interest will generally comprise the nucleotides that are of interest (i.e., may span a region of the genome comprising the variant of interest). In some cases, the genomic region of interest may comprise one or more clinically actionable variants. The genomic region of interest may be within the coding sequence of a gene (e.g., an exon), may be within a non-coding region (e.g., an intron), or both. The genomic region of interest may comprise one or more structural variants (e.g., translocations, copy number variations) and/or nucleotide variants. In some cases, the genomic region of interest is investigated to determine the presence or absence of a genetic variant. In some cases, a user of the methods selects a genomic region of interest to be queried. In some cases, a user of the method selects the genetic variant to be queried and the genomic region of interest is determined by the selection. Put another way, the selection of the genetic variant may define the genomic region of interest.

The methods may comprise comparing a quality score to a threshold value. A threshold value may be used as a cut-off value by which to assess a quality score. A threshold value can be predetermined or preset. In some cases, the threshold value is empirically determined. In some cases, the threshold value is determined by a user of the methods. The threshold value may be adjustable such that a user of the methods can change or alter the threshold value. In some cases, the threshold value may be more stringent or less stringent based on the needs of the user. The threshold value may be a value by which a quality score can be compared to determine the accuracy of the data. The threshold value may be a value above which a quality score indicates a certain level of confidence in the accuracy of the variant call. For example, a quality score above a threshold value may indicate a 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.9%, 99.99%, 99.999%, or 100% confidence in the accuracy of a variant call. The threshold value may be a value below which a quality score indicates a certain level of confidence in the inaccuracy of the variant call. For example, a quality score below a threshold value may indicate a 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.9%, 99.99%, 99.999%, or 100% confidence in the inaccuracy of a variant call.

In some cases, a threshold value may correspond to a read depth. In this example, a read depth of each genomic region of interest can be compared to the threshold value. A genomic region of interest with a read depth exceeding the threshold value may be identified as having “sufficient” coverage and a genomic region of interest with a read depth below the threshold value may be identified as having “insufficient” coverage. A genomic region of interest identified as having “insufficient” coverage may be e.g., re-sequenced. A threshold value based on read depth can include 1×, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 60×, 70×, 80×, 90×, 100×, 200×, 300×, 400×, 500×, 600×, 700×, 800×, 900×, 1000×, or greater. In one case, the threshold value is 10×. In another case, the threshold value is 20×. In another case, the threshold value is 30×. In another case, the threshold value is 40×. In yet another case, the threshold value is 50×. In yet another case, the threshold value is 100×.

A quality score can be utilized to classify one or more genetic variants. Classifying one or more genetic variants may comprise comparing the quality score of each of the one or more genetic variants to the threshold value. It should be understood that any value, number, letter, word, or score can be utilized to classify a genetic variant, as long as the classification represents the class to which the genetic variant has been assigned. For example, an arbitrary number (e.g., 10) and a word (“present”) can represent the same concept (i.e., that a variant is “present”). In one example, the classification system described herein may determine whether the quality score for a given genetic variant (or genomic region) is “sufficient” or “insufficient” to proceed with analysis of the data. In some cases, genetic variants may be classified as “present”, “absent”, or “indeterminate”. A genetic variant may be classified as present, for example, if the genetic variant is present (i.e., variant is “called”) and the quality score of the called base (or a genomic region comprising the called base) is greater than the threshold value. A classification of “present” can indicate that a genetic variant is positively identified as being present with an accuracy of at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.9%, 99.99%, 99.999%, or 100%. In other cases, a genetic variant may be classified as absent, for example, if the genetic variant is absent (i.e., one or more nucleotide other than the genetic variant is called) and the quality score of the called base (or a genomic region comprising the called base) is greater than the threshold value. A classification of “absent” can indicate that a genetic variant is positively identified as being absent with an accuracy of at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.9%, 99.99%, 99.999%, or 100%. In some cases, a quality score may comprise a confidence score. A confidence score may be 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In some cases, a genetic variant may be classified as “indeterminate” if the quality score of the called base (or a genomic region comprising the called base) is lower than the threshold value. An “indeterminate” classification can indicate that the quality of the data used to support the called base is too low such that the accuracy of the call cannot be determined. The methods provided herein can be useful to distinguish between variants that cannot be called due to low quality data and variants that are not present.

In some cases, genetic variants can be organized by variant class (e.g., EGFR-activating mutation, BRAF-inactivating mutation). A variant class can comprise one or more genetic variants with similar function (e.g., gain of function of EGFR). A variant class can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more genetic variants. In some cases, a variant class as a group can be assigned a classification. A variant class can be assigned a classification of “present” or “absent” based on similar criteria described above. In some cases, a variant class classification can correspond to the classification of a single genetic variant within that variant class. For example, if even one genetic variant of the EGFR-activating variant class (in a group of a plurality of EGFR-activating variants) is assigned a classification of “present,” the EGFR-activating variant class as a group is assigned a classification of “present.” In some cases, more than one genetic variant within a variant class may need to be assigned a classification of “present” in order for the variant class as a group to be assigned a classification of “present.”

An “indeterminate” classification can indicate that at least one modification be made to a sequencing protocol. A modification to a sequencing protocol can include any modification to the sample preparation, sample processing, or sequencing steps. In some cases, a modification to a sequencing protocol may be an optimization of a sequencing protocol (i.e., to optimize the results of the sequencing methods). A modification can be made to at least one of a probe, a primer, or a reaction condition. In a particular example, a clinically actionable variant may be found within a genomic region that is problematic (e.g., a GC-rich region). These regions may result in an “indeterminate” classification for clinically actionable variants within these regions. The sequencing protocol utilized to generate the sequencing data can be analyzed and a modification can be made to the sequencing protocol (e.g., a modified capture probe that hybridizes to a sequence outside of the GC-rich region). In some cases, the sequencing protocol is a target-enrichment protocol comprising at least one of target-specific primers and target-specific probes. In this example, a modification can be made to at least one of the target-specific primers or target-specific probes.

The methods can further provide for translating regions of insufficient coverage or with low quality scores into genomic coordinates. Genomic coordinates allow the user of the methods to pinpoint the exact location of the genomic regions of interest or the genetic variant. Genomic coordinates may comprise the chromosome number (e.g., chromosome 10) as well as the exact location of the region or variant on that chromosome. Genomic coordinates can provide the exact addressable position of a region or a variant on a chromosome (i.e., a genetic address). Genomic coordinates can be utilized in the methods herein. For example, the genomic coordinates for modified primers or probes can be provided to the user for e.g., ordering modified primers or probes from a vendor.

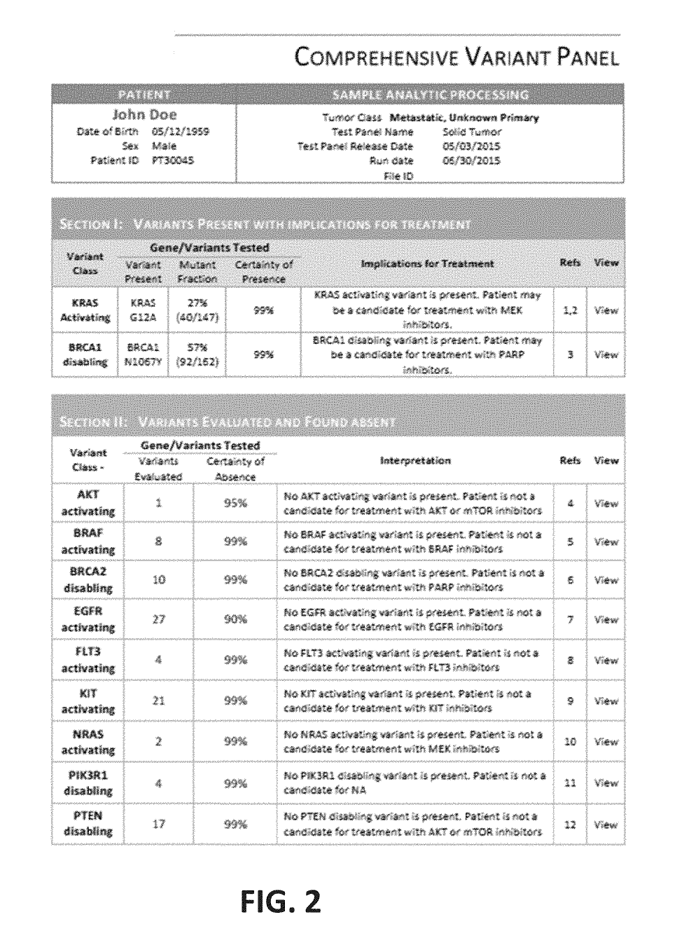

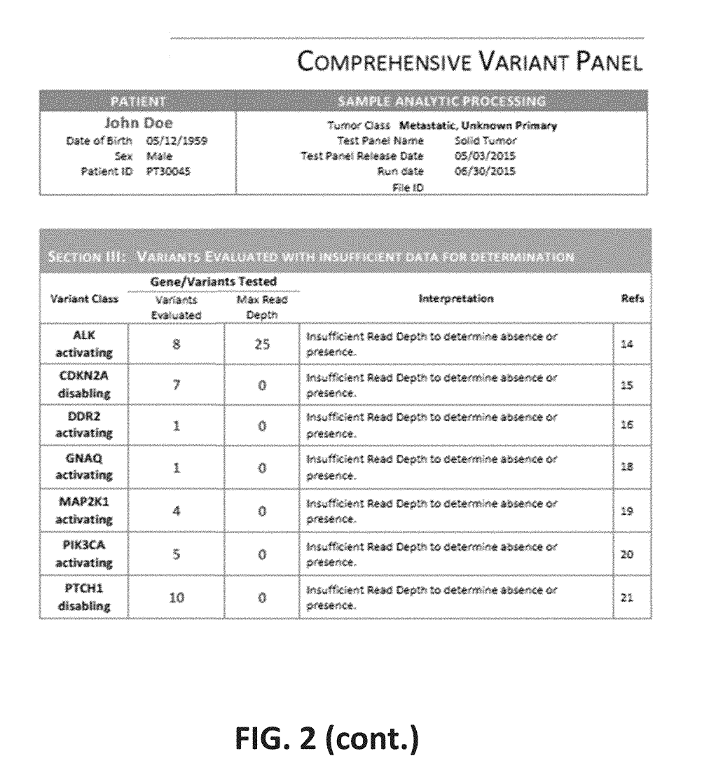

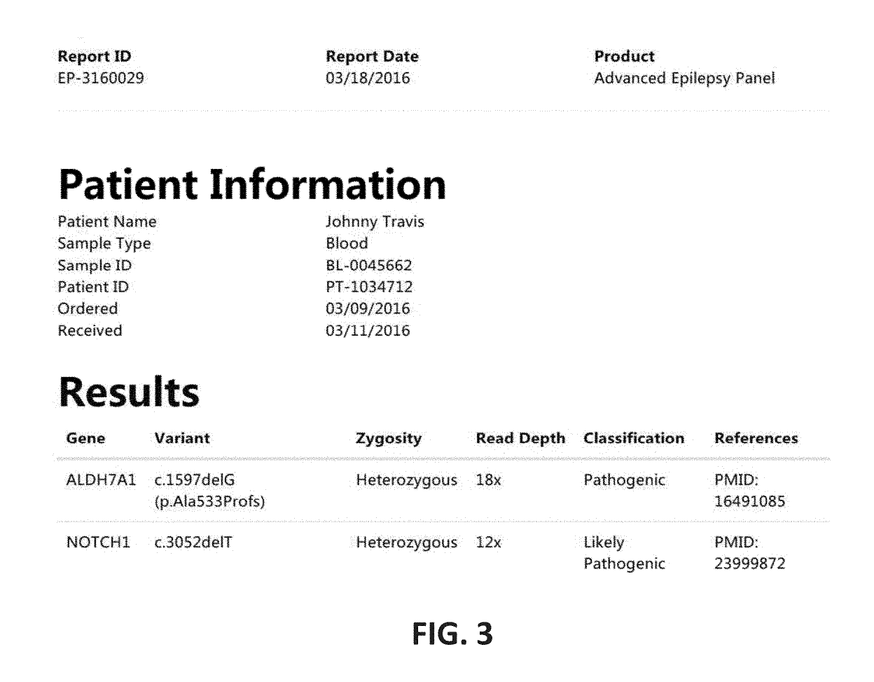

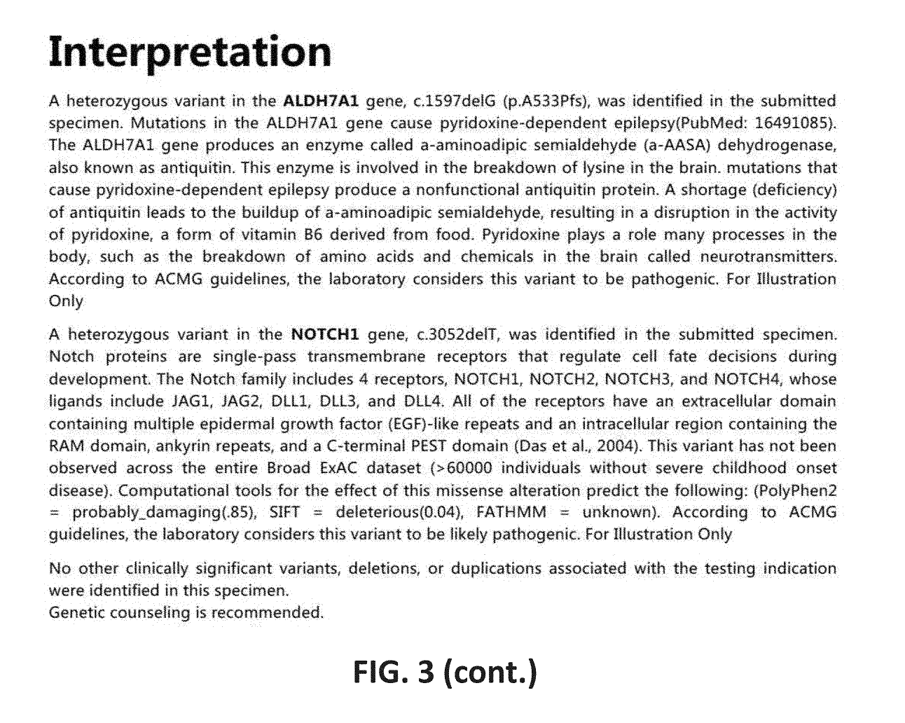

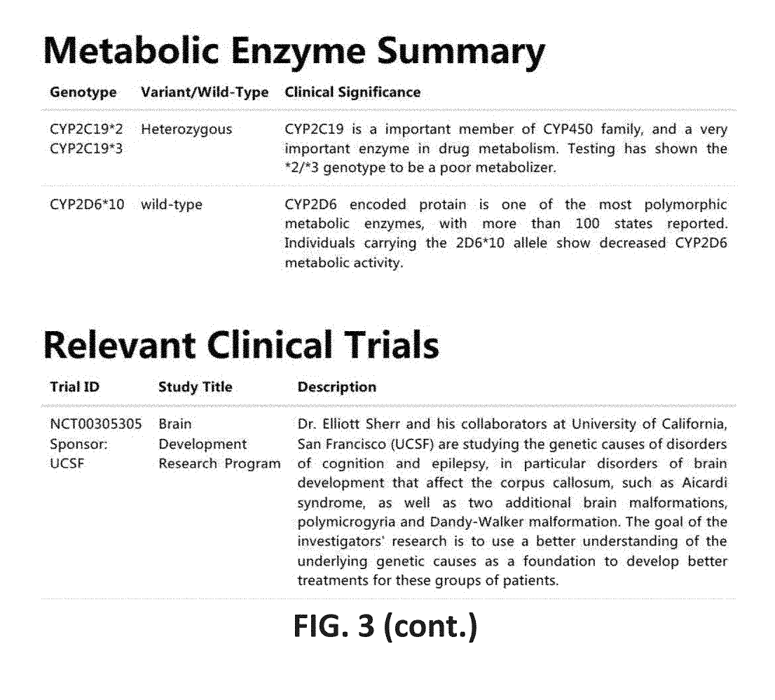

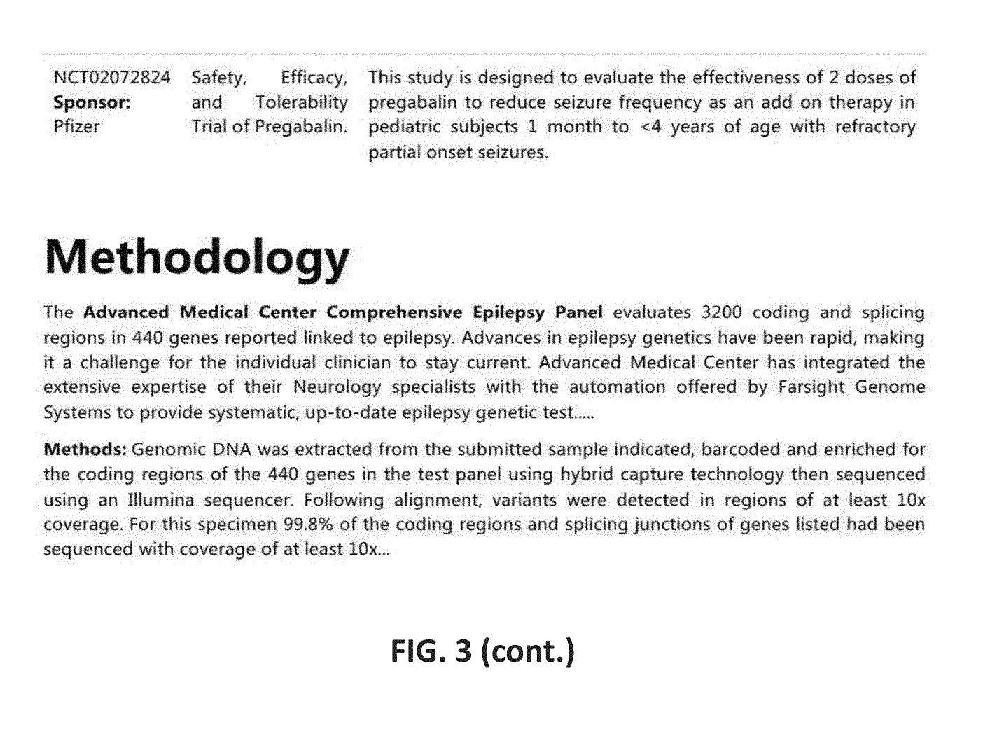

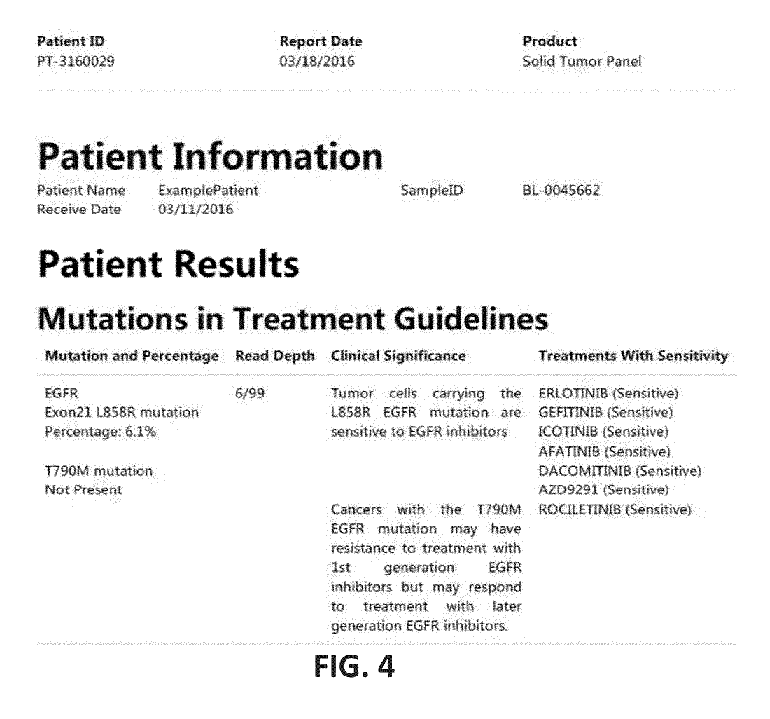

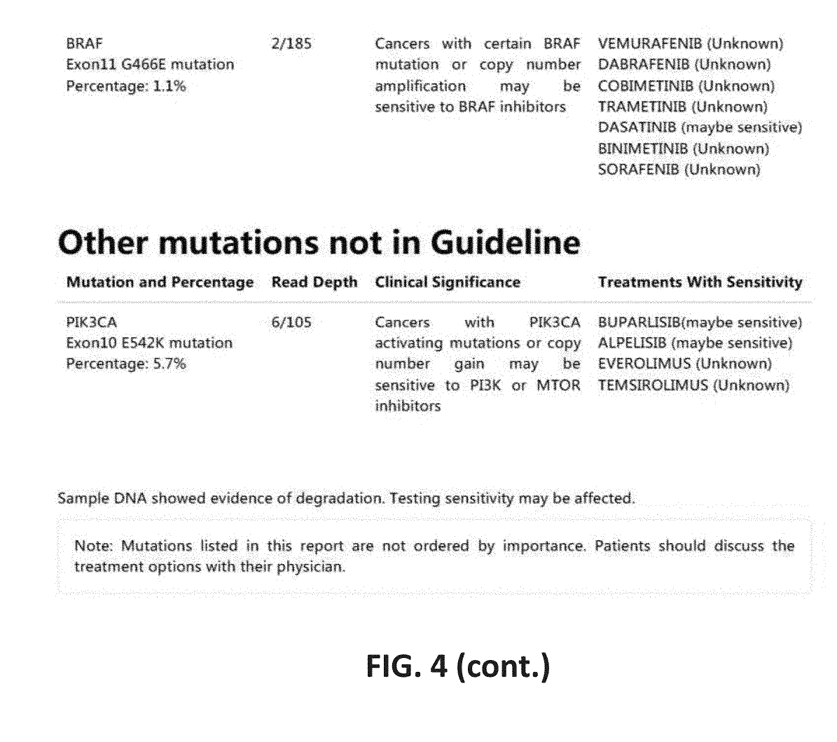

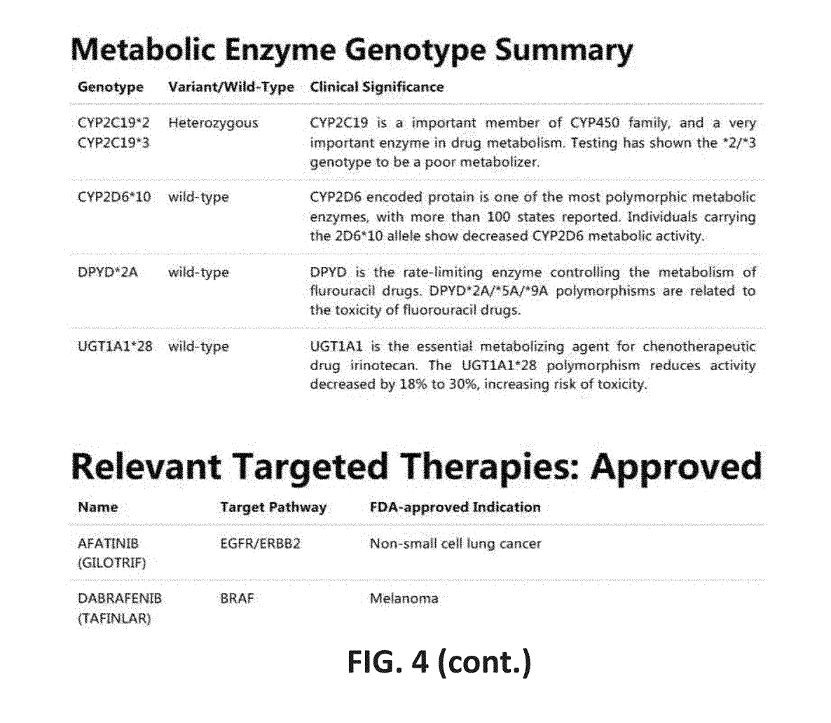

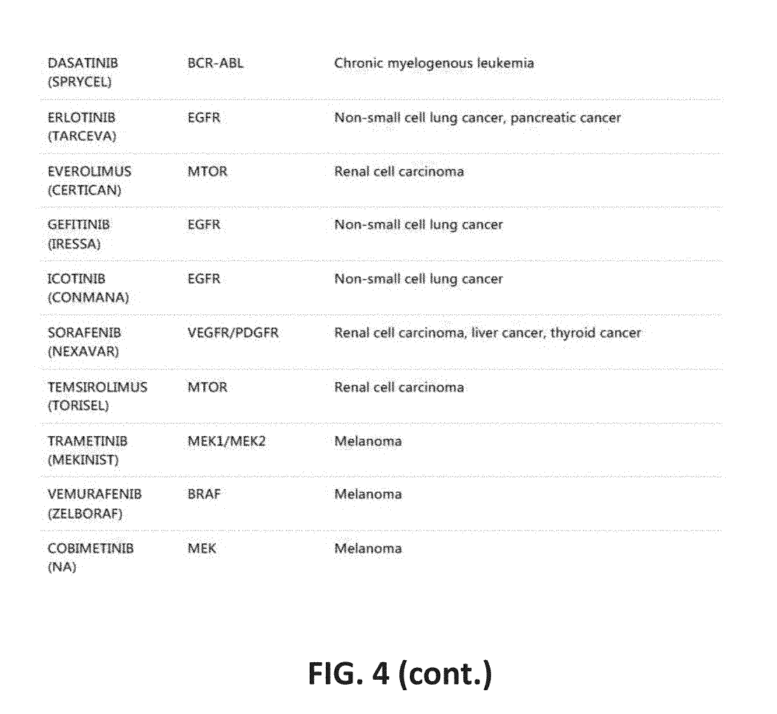

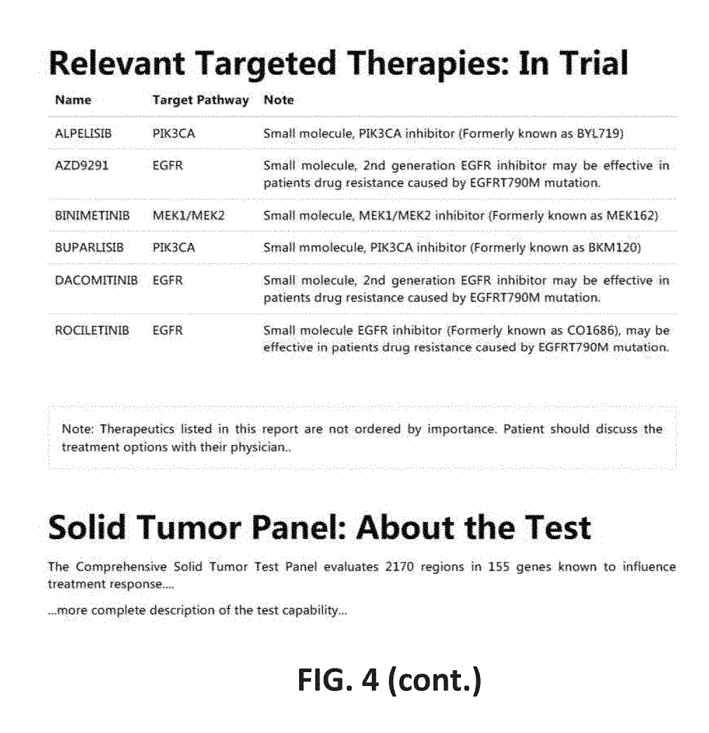

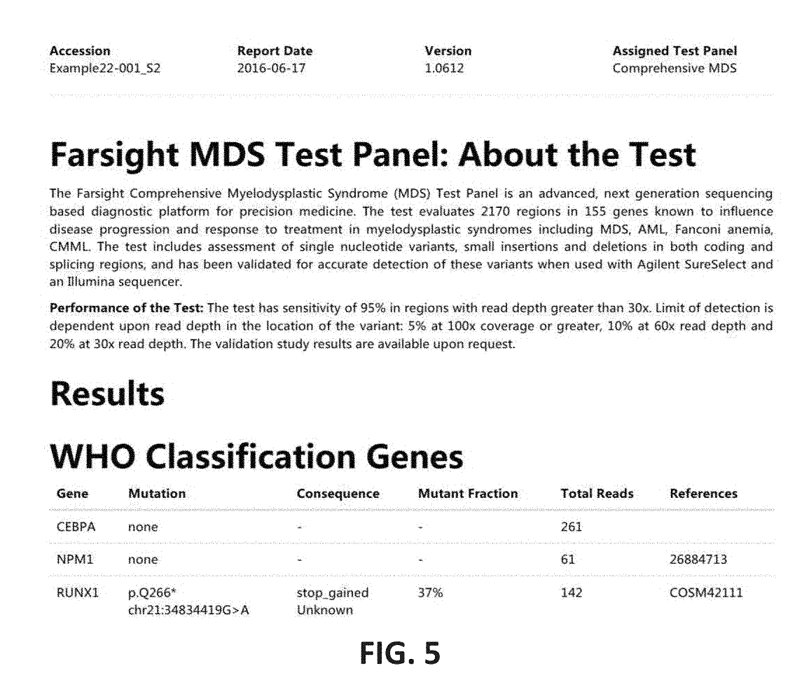

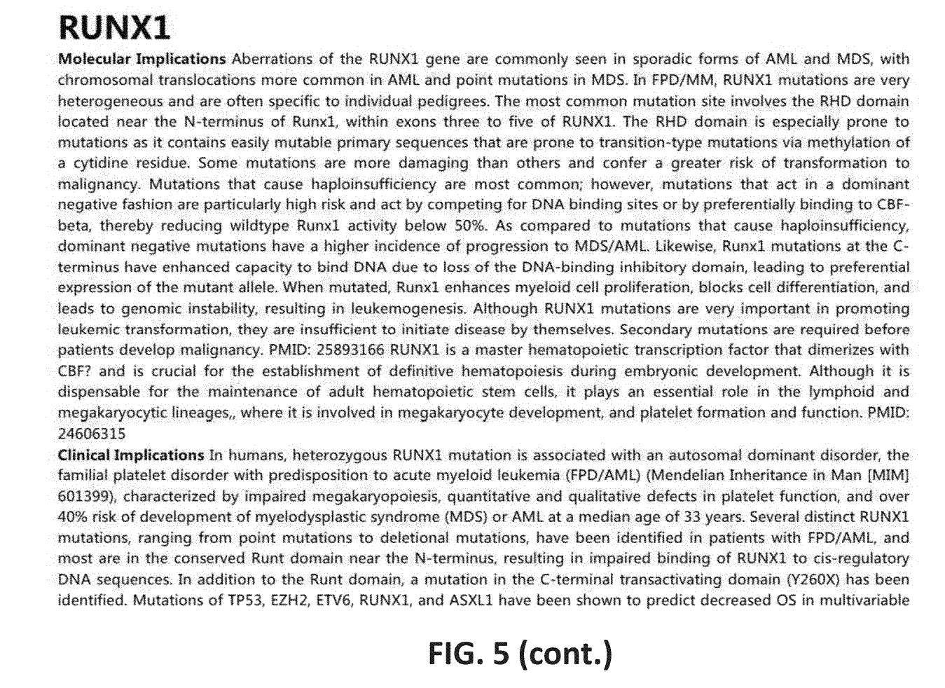

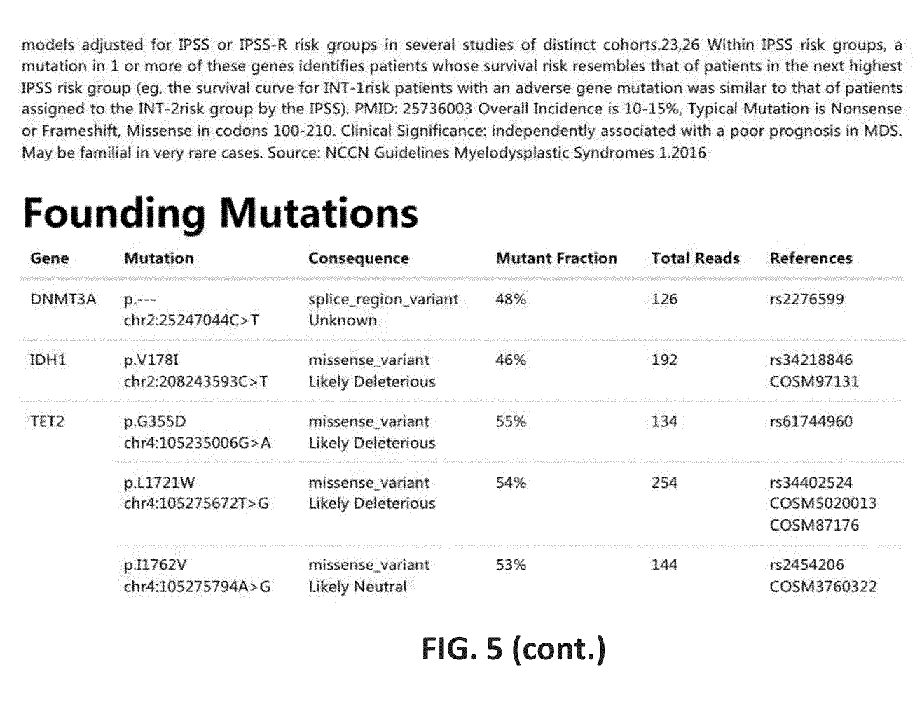

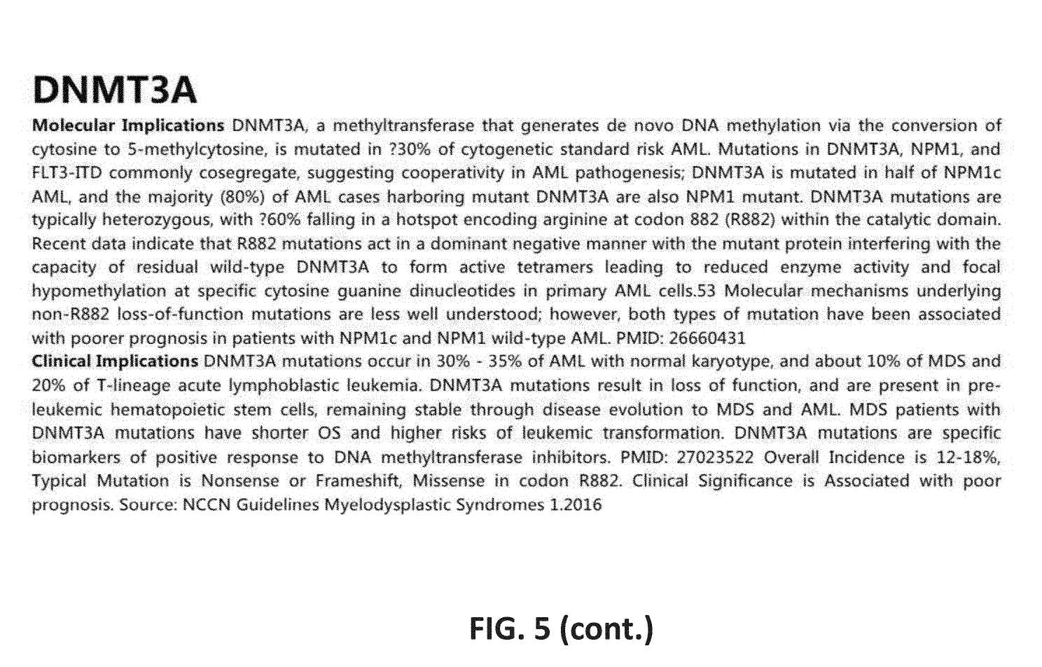

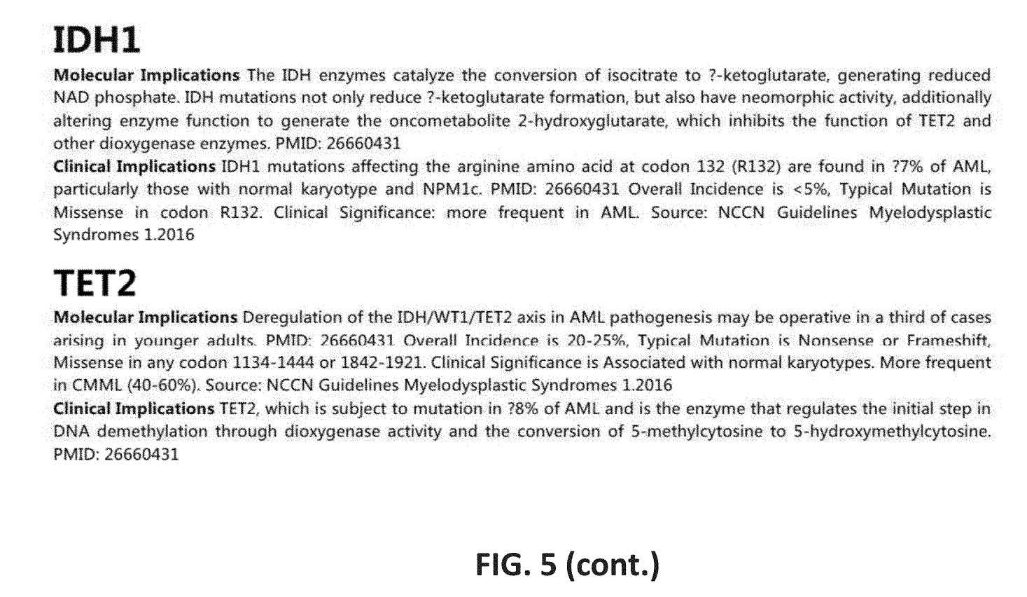

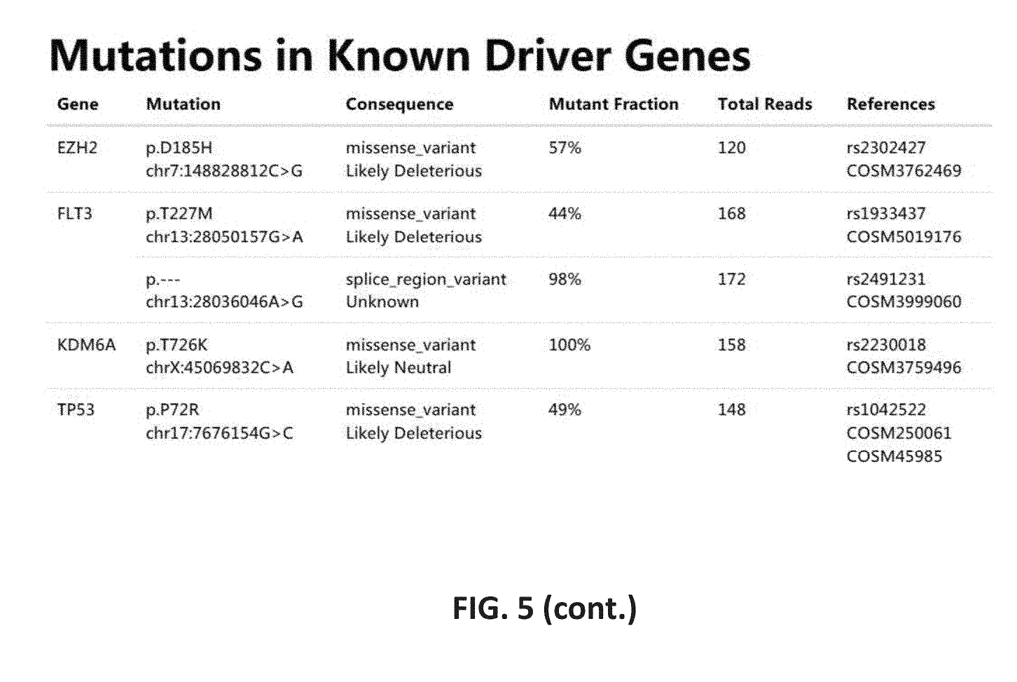

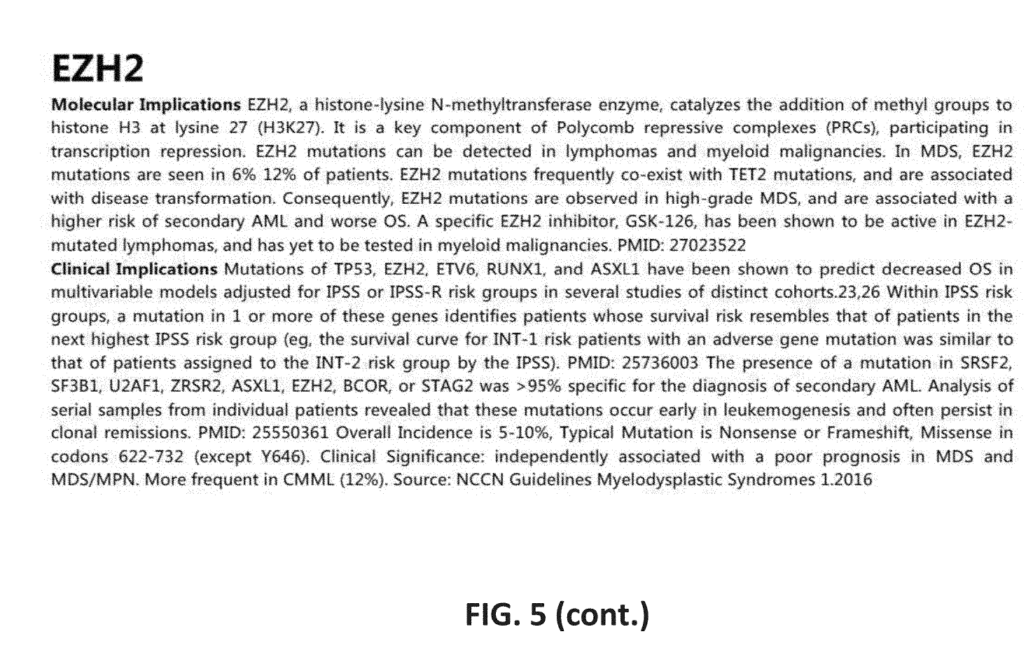

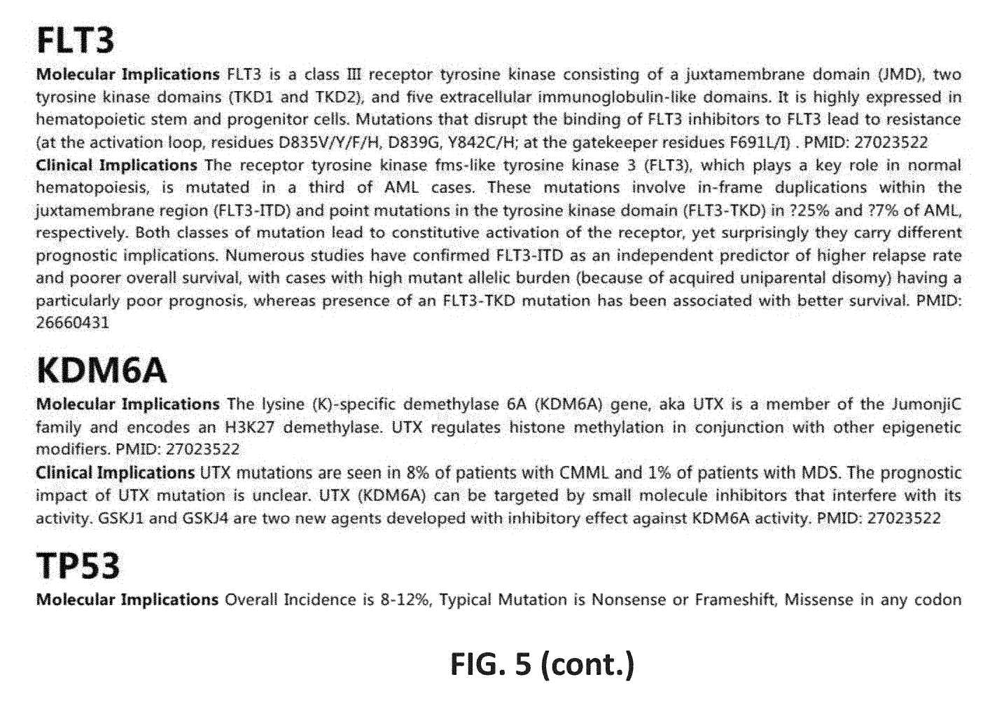

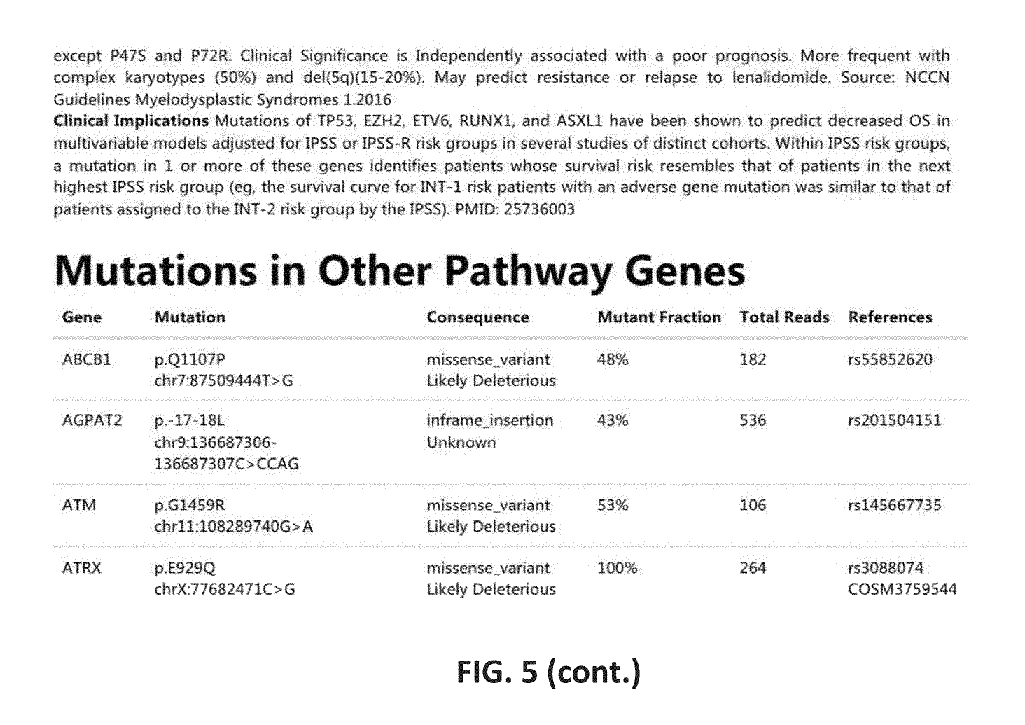

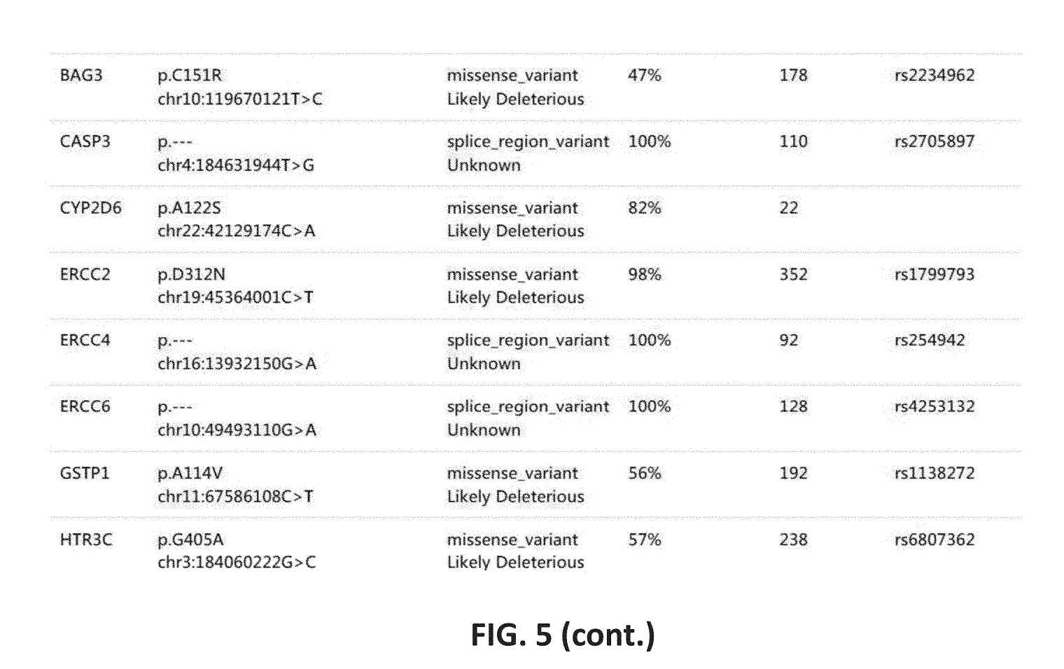

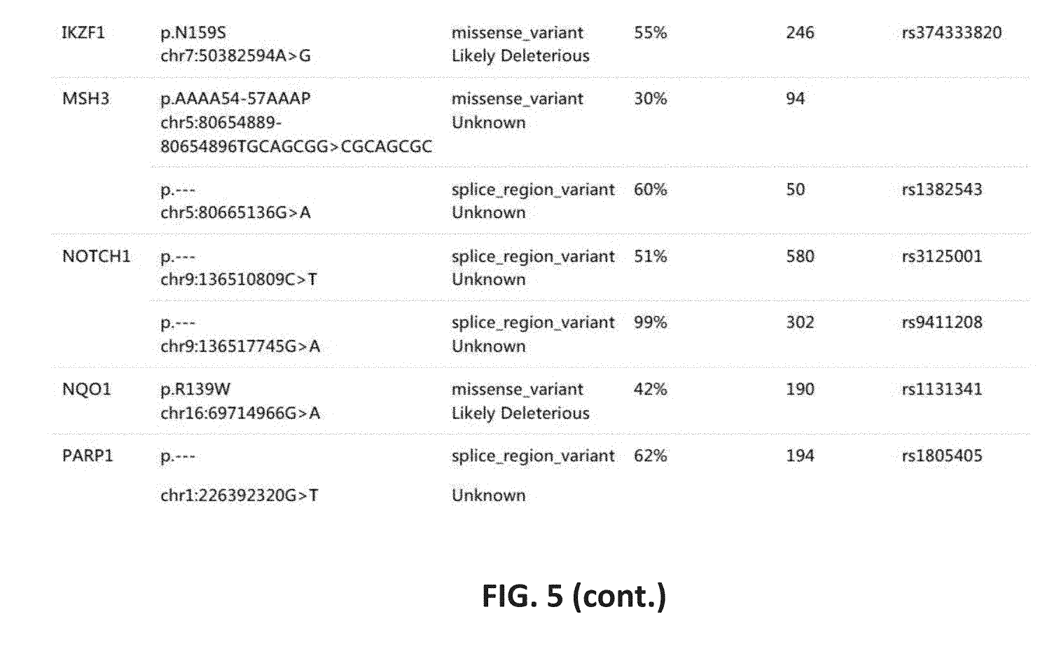

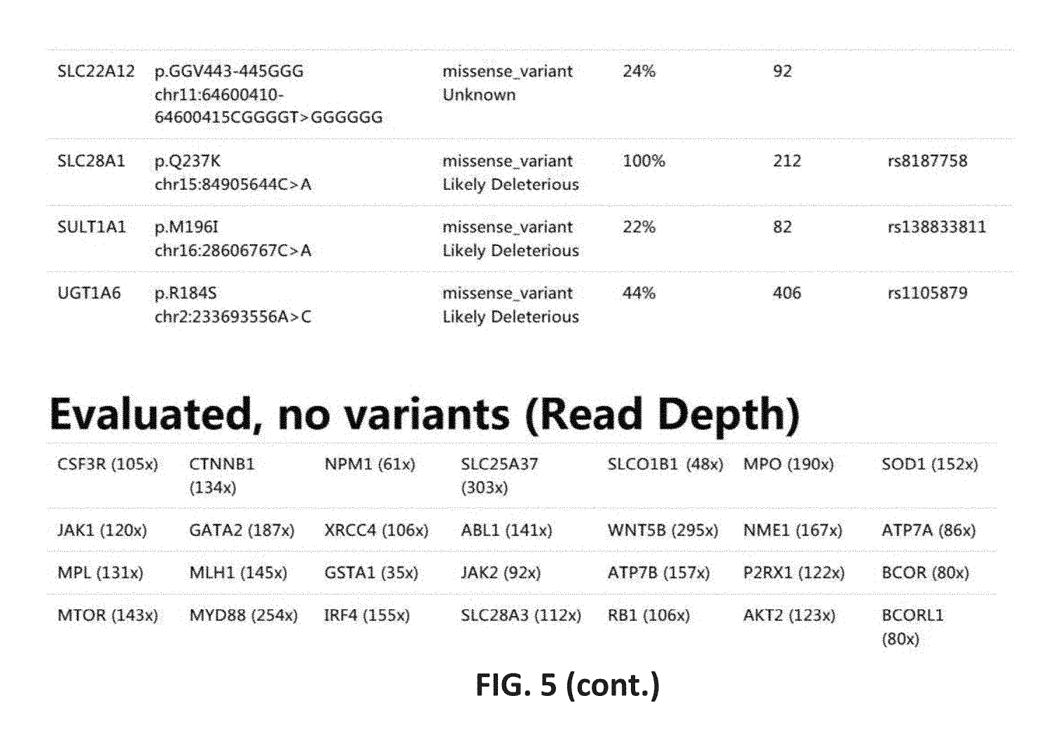

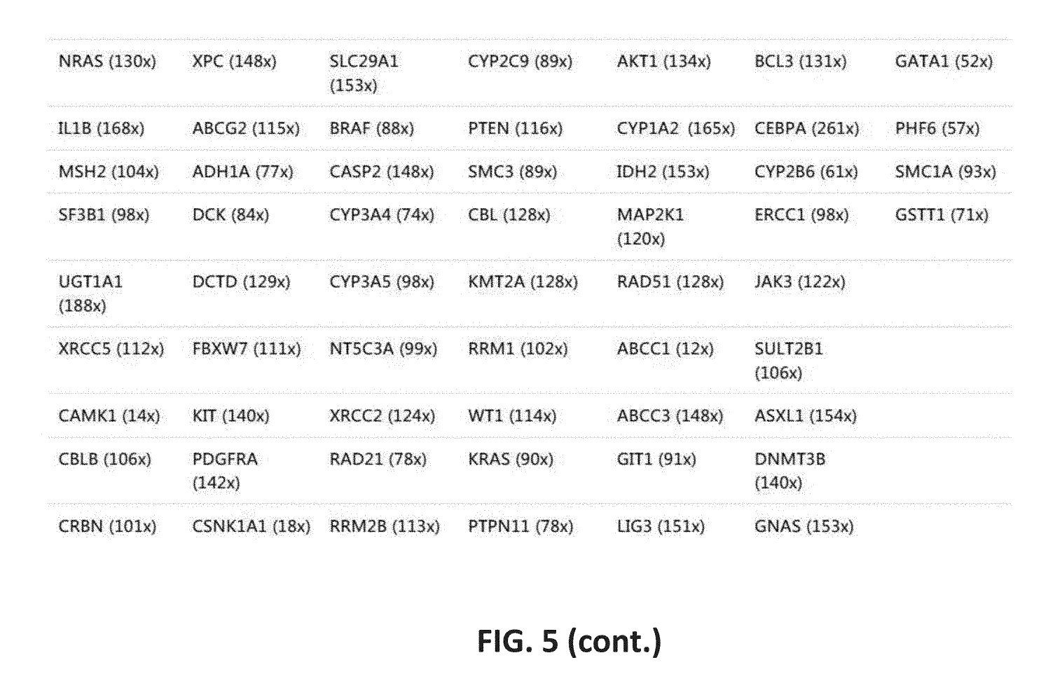

The methods further provide for generating a report wherein the report can identify the classified genetic variant. Examples of reports that can be generated by the methods and systems disclosed herein are depicted in FIGS. 2-5. A report can be any means by which the results of the methods described herein are relayed to an end-user. The report can be displayed on a screen or electronic display or can be printed on e.g., a sheet of paper. In some cases, the report is transmitted over a network. In some cases, the network is the Internet. In some cases, the report can be transmitted as a data representation in JSON, HL7 or similar format for transformation into an electronic medical record. In some cases, the report may be generated manually. In other cases, the report may be generated automatically. In some cases, the report may be generated in real-time. The report can identify the classified genetic variant, for one or more of the variants in the test panel. For example, the report can identify at least one genetic variant classified as “present,” at least one genetic variant classified as “absent,” at least one variant classified as “indeterminate,” or any combination thereof. In some examples, the report can identify at least one classification of a variant class. In the example of an “indeterminate” classification, the report can suggest or recommend a modification to a sequencing protocol as described above. The report can further provide additional information about the classified genetic variants. In some cases, the report can provide a treatment plan or treatment recommendation based on the results of the test. In this example, the presence or absence of a variant can indicate that the patient may be responsive or refractory to a particular therapy. The report can present this information to the end-user (e.g., a patient, a healthcare provider, or a clinical laboratory). In some cases, the report can be provided to a mobile device, smartphone, tablet or personal health monitor or other network enabled device. In some cases, a treatment decision can be made based on the information in the report. In some cases, a treatment can be administered to a subject based on the report. In some examples, the patient may be receiving a therapy for a disease prior to ordering the genetic test. The report may indicate that a genetic variant is present and that the current treatment regimen should be ceased and a new treatment regimen be administered. In some cases, the patient is tested prior to receiving treatment and further tests are ordered during the course of the treatment. In this example, the patient is monitored for the presence or absence of de novo genetic variants that may indicate the current treatment regimen is no longer effective as a therapy for that patient. The report may further indicate or recommend a different course of treatment based on the presence or absence of de novo genetic variants. The report can provide additional information including, without limitation, genomic coordinates of the variant or genomic region of interest, images that locate the variant within the functional region of the protein, images that show the aligned read stack in the region of the variant, attachments or links (i.e., hyperlinks) to references (i.e., scientific literature) related to the variant of interest, the clinical evidence supporting the treatment recommendations, guidelines that support clinical use of the variant, or reimbursement codes related to the diagnosis or treatment, or any other useful information.

The methods further provide for receiving a second data input. In some cases, the second data input comprises second sequencing data. The second sequencing data can be different sequencing data to that which was originally submitted. Any methods described herein with regards to sample preparation, sample processing, and sequencing can be utilized to generate the second sequencing data. In some cases, the second sequencing data can be sequencing data generated from a modified sequencing protocol. The modified sequencing protocol can be a modified sequencing protocol generated from the methods described above. In this case, the second sequencing data can be optimized such that a quality score of a genomic region of interest is improved as compared to a prior iteration of the methods. These methods may be particularly suited to reanalyzing regions of interest that are classified as “indeterminate” (i.e., regions of interest with a quality score below the threshold value). In this example, the quality score of the reanalyzed region of interest may exceed the threshold value such that a classification of “present” or “absent” can be assigned to the variant.

In some cases, the methods further provide for requerying the sequencing data to determine a presence or an absence of one or more additional genetic variants. Requerying may involve reanalyzing previously analyzed sequencing data (i.e., without receiving additional sequencing data). In this case, a quality score can be assigned to each of one or more genomic regions including the one or more additional genetic variants. The quality score may be classified as sufficient if the quality score is greater than a predetermined threshold and the quality score may be classified as insufficient if the quality score is lower than a predetermined threshold.

In another aspect of the disclosure, a method is provided for evaluating the accuracy of a previously analyzed sequencing data set. For example, a sequencing data set may have been previously analyzed and reported in a scientific paper or article. In some cases, the analysis may report an average depth of coverage for the overall sequencing data set, however, local depth of coverage may be unknown. In some cases, the original analysis may report the presence or absence of one or more genetic variants identified from the sequencing data set. In some cases, the methods involve determining a quality score for one or more genomic regions, wherein the one or more genomic regions include at least one of the one or more genetic variants that have been previously analyzed. Any of the methods provided herein may be utilized to perform the analysis. For example, a quality score may be assigned to each genomic region being investigated. In some cases, the quality score is a depth of coverage. The methods may further involve evaluating the accuracy of the original analysis by identifying each genetic variant as being accurately called or inaccurately called based on the quality score. For example, if the original analysis identified a genetic variant within a genomic region that has a quality score less than a predetermined threshold, the evaluating may involve identifying the original analysis as inaccurate. Vice versa, if the original analysis identified a genetic variant within a genomic region that has a quality score greater than a predetermined threshold, the evaluating may involve identifying the original analysis as accurate. Methods previously disclosed herein for identifying the presence or absence of genetic variants may be used to supplement or enhance the original analysis, for example, to correct an inaccurate analysis. In some cases, if the original analysis for a genetic variant is identified as inaccurate, a modification to a sequencing protocol may be recommended.

In a particular aspect of the disclosure, a method is provided comprising: (a) receiving a data input comprising sequencing data generated from a nucleic acid sample from a subject, wherein, prior to the receiving, the sequencing data has been analyzed and a presence or absence of one or more genetic variants has been identified, thereby generating an original analysis of the sequencing data; (b) assigning a quality score to each of one or more genomic regions of the sequencing data, the one or more genomic regions comprising at least one of the one or more genetic variants, wherein the assigning is performed by a computer processor; (c) evaluating the original analysis of the one or more genetic variants based on the quality scores, and (d) outputting a result based on the evaluating, wherein the evaluating further comprises identifying the original analysis for a genetic variant of the one or more genetic variants as accurate if the quality score for the genomic region comprising the genetic variant is greater than a predetermined threshold, and wherein the evaluating further comprises identifying the original analysis for a genetic variant of the one or more genetic variants as inaccurate if the quality score for the genomic region comprising the genetic variant is less than a predetermined threshold.

### Processing Steps

Nucleic acids can be processed and/or analyzed by any method known to those skilled in the art. In some cases, the methods disclosed herein may be performed by conducting one or more enrichment reactions on one or more nucleic acid molecules in a sample. The enrichment reactions may comprise contacting a sample with one or more beads or bead sets. The enrichment reactions may comprise one or more hybridization reactions. The one or more hybridization reactions may comprise the use of one or more capture probes. The one or more capture probes may comprise one or more target-specific capture probes. The target-specific capture probes may hybridize to a nucleic acid sequence in an exon of a gene. The enrichment reactions may further comprise isolation and/or purification of one or more hybridized nucleic acid molecules. The enrichment reactions may comprise whole exome enrichment. The enrichment reactions may comprise targeted enrichment. The enrichment reaction may be performed with the use of a kit or a panel, commercially available examples include, without limitation, Agilent Whole Exome SureSelect, NuGEN Ovation Fusion Panel, and Illumina TruSight Cancer Panel.

In some cases, the enrichment reactions may comprise one or more amplification reactions. The one or more amplification reactions may comprise amplifying a nucleic acid sequence by e.g., polymerase chain reaction. The amplifying may comprise the use of one or more sets of primers. The one or more sets of primers can be target-specific primers to amplify a targeted nucleic acid sequence. The one or more sets of target-specific primers may hybridize to a nucleic acid sequence in an exon of a gene. The amplified nucleic acid sequences may be further purified, isolated, extracted, and the like. In some cases, one or more barcodes and/or adaptors can be appended to the amplified nucleic acid sequences. The one or more barcodes and/or adaptors can be barcodes and/or adaptors useful in e.g., a sequencing reaction.

In some cases, the nucleic acids are sequenced to generate sequencing data. Sequencing data can be generated by any known sequencing method. The sequencing methods may comprise capillary sequencing, next generation sequencing, Sanger sequencing, sequencing by synthesis, single molecule nanopore sequencing, sequencing by ligation, sequencing by hybridization, sequencing by nanopore current restriction, or a combination thereof. Sequencing by synthesis may comprise reversible terminator sequencing, processive single molecule sequencing, sequential nucleotide flow sequencing, or a combination thereof. Sequential nucleotide flow sequencing may comprise pyrosequencing, pH-mediated sequencing, semiconductor sequencing or a combination thereof. Conducting one or more sequencing reactions comprises untargeted sequencing (i.e., whole genome sequencing) or targeted sequencing (i.e., exome sequencing).

The sequencing methods may comprise Maxim-Gilbert, chain-termination or high-throughput systems. Alternatively, or additionally, the sequencing methods may comprise Helioscope™ single molecule sequencing, Nanopore DNA sequencing, Lynx Therapeutics' Massively Parallel Signature Sequencing (MPSS), 454 pyrosequencing, Single Molecule real time (RNAP) sequencing, Illumina (Solexa) sequencing, SOLiD sequencing, Ion Torrent™, Ion semiconductor sequencing, Single Molecule SMRT™ sequencing, Polony sequencing, DNA nanoball sequencing, VisiGen Biotechnologies approach, or a combination thereof. Alternatively, or additionally, the sequencing methods can comprise one or more sequencing platforms, including, but not limited to, Genome Analyzer IN, HiSeq, NextSeq, and MiSeq offered by Illumina, Single Molecule Real Time (SMRT™) technology, such as the PacBio RS system offered by Pacific Biosciences (California) and the Solexa Sequencer, True Single Molecule Sequencing (tSMS™) technology such as the HeliScope™ Sequencer offered by Helicos Inc. (Cambridge, Mass.), nanopore-based sequencing platforms developed by Genia Technologies, Inc., and the Oxford Nanopore MinION.

Sequencing data can be received (e.g., by a computer processor coupled to a computer memory source) as a data input. Sequencing data can be received as a text-based or binary file format representing nucleotide sequences. Sequencing data can be received as, for example, SRA, CRAM, FASTA, SAM, BAM, or FASTQ file formats. In particular examples, the sequencing data is received in a FASTQ file format. FASTQ file formats store nucleotide sequencing data along with the corresponding quality data.

### Clinically Actionable Variants

The methods and systems disclosed herein can be utilized to identify one or more clinically actionable variants. In some cases, the methods and systems can be used to classify one or more clinically actionable variants. The clinically actionable variant can be in a coding region of a gene or can be in a non-coding region of the genome. The non-coding region of the genome can be a regulatory region of the gene. The clinically actionable variant can be in an exon of a gene or can be in an intron of a gene. A clinically actionable variant may alter the expression of the gene or may alter the function of the gene product (i.e., the function of the protein). A clinically actionable variant can regulate a gene involved in a disease. In particular examples, the clinically actionable variant alters the expression of or the function of a known cancer gene. In some cases, the clinically actionable variant alters the response of a protein to a therapy. For example, a clinically actionable variant may indicate that a protein is refractory to a specific therapy (e.g., a variant in an antigen such that an antibody therapy no longer recognizes the antigen).

In particular cases, a clinically actionable variant can be identified and/or classified in a subject or patient is suffering from cancer. In one example, the clinically actionable variant can be an activating or an inactivating mutation in a target gene. In some cases, the clinically actionable variant may be an activating mutation in a gene known to affect the responsiveness of a tumor to a therapy or in a proto-oncogene is present or absent. An “activating mutation” can be any genetic variant that results in a new function of or an increased activity level of (i.e., “gain-of-function”) a protein. An activating mutation can be a large-scale variation such as an amplification, insertion or translocation, or can be a small-scale variation such as a point mutation. In some cases, the activating mutation is in a target gene. In other cases, the activating mutation is in a regulatory region or non-coding region of a target gene. In some cases, the presence of an activating mutation can indicate that a subject is a candidate for a specific therapy or treatment. In other cases, the absence of an activating mutation can indicate that a subject is not a candidate for a specific therapy or treatment. In some cases, the clinically actionable variant can be an inactivating mutation in a gene known to affect the responsiveness of a tumor to a therapy or in a tumor suppressor gene is present or absent. An “inactivating mutation” can be any genetic variant that results in a loss of function or a decreased activity level of a protein. An inactivating mutation can be a large-scale variation such as a deletion or copy number loss, or can be a small-scale variation such as a point mutation. In some cases, the inactivating mutation is in a target gene. In other cases, the inactivating mutation is in a regulatory region or non-coding region of a target gene. In some cases, a subject may have one or more activating and/or inactivating mutations in one or more target genes.

In some cases, the clinically actionable variant may be a mutation in a gene or regulatory region of a gene that alters the responsiveness of the gene product (i.e., protein) to a therapy. In one example, the clinically actionable variant is a mutation that can affect a metabolic gene and can increase or decrease the responsiveness to a given drug therapy. A metabolic gene can be a gene that alters the pharmacogenomics of a therapeutic drug. For example, the presence of a variant in the UGT1A1 gene (e.g., UGT1A1*28 and/or UGT1A7*3) may suggest that the subject is at higher risk of severe hematologic toxicity when treated with irinotecan (CAMPTOSAR). In another example, the presence of a specific combination of variants in the cytochrome P450 2D6 enzyme may suggest a subject is not recommended to be treated with tamoxifen.

In some cases, the clinically actionable variant is a mutation that affects a transport gene. A transport gene can be any gene that controls influx or efflux across cell membranes (i.e., channels, pumps, transporters). In a non-limiting example, the presence of a variant in the ABC transporter gene, ABCC3 (e.g., rs4148416) can indicate that an osteosarcoma patient may exhibit poor response to treatment with cisplatin, cyclophosphamide, doxorubicin, methotrexate, or vincristine. In another non-limiting example, the presence of a variant in the ABCB1 gene (e.g., rs1045642) can be associated with lower survival in Asian metastatic breast cancer patients treated with paclitaxel. In yet another non-limiting example, the presence of the rs316019 variant in SLC22A2 can be associated with an increased risk of nephrotoxicity in patients treated with cisplatin.

In some cases, the clinically actionable variant can be a variant that is associated with an unexpected or exceptional response to a given drug therapy. In a non-limiting example, an advanced stage cancer patient with a variant in mTOR (e.g., E2419K and E2014K) may demonstrate an exceptional response to treatment with everolimus. In another non-limiting example, a metastatic small cell lung cancer patient with the variant L1237F in the RAD50 gene may demonstrate an exceptional response to treatment with AZD7762 and irinotecan. In another non-limiting example, a hepatocellular carcinoma patient with the rs2257212 variant in the SLC15A2 gene may demonstrate an exceptional response to treatment with sorafenib.

In some cases, the clinically actionable variant can affect a DNA repair gene. In a non-limiting example, a patient with a solid tumor and a variant in the ERCC1 gene may demonstrate an improved response to treatment with platinum-based compounds. In another non-limiting example, the presence of a variant in the XRCC1 gene may indicate that a patient may demonstrate an increased response to fluorouracil, carboplatin, cisplatin, oxaliplatin, and other platinum-based compounds.

In some cases, the clinically actionable variant is associated with increased toxicity or other severe adverse events. In a non-limiting example, a patient homozygous for DPYD*2A, DPYD*13 or rs67376798 can indicate that the patient may experience severe toxicity when treated with fluoropyrimidines (i.e., 5-fluorouracil, capecitabine or tegafur). In another non-limiting example, the presence of the TPMT*3B or TPMT*3C variants can indicate that a child treated with cisplatin, mercaptopurine, or thioguanine may be at an increased risk of ototoxicity. In yet another non-limiting example, a patient with G6PD deficiency may experience severe adverse side effects when treated with doxorubicin, daunorubicin, rasburicase, or dabrafenib.

In some cases, the clinically actionable variant is located within a gene that is not known to play a direct role in a given disease. For example, a clinically actionable variant can be located within a gene that does not play a direct role in cancer but can alter a response of the patient to a given cancer treatment. It should be understood, then, that a clinically actionable variant as envisioned herein is any variant that can indicate or predict a clinical outcome in a subject.

In some cases, the clinically actionable variant is in a gene that is known to cause or contribute to the pathogenesis of cancer. In some cases, the disease is cancer. Non-limiting examples of genes known to cause or contribute to the pathology of cancer can include: ABCA1, ABCC3, ABCG2, ABL1, ACSL6, ADA, ADCY9, ADM, AGAP2, AIP, AKT1, AKT2, AKT3, ALK, ALOX12B, ANAPC5, APC, APC2, APCDD1, APEX1, AR, ARAF, ARFRP1, ARID1A, ARID1B, ARID2, ARID5B, ASXL1, ASXL2, ATM, ATR, ATRX, AURKA, AURKB, AXIN1, AXIN2, AXL, B2M, BACH1, BAI3, BAP1, BARD1, BAX, BBC3, BCL11A, BCL2, BCL2L1, BCL2L11, BCL2L2, BCL3, BCL6, BCOR, BCORL1, BCR, BIRC3, BIRC5, BIRC6, BLM, BMP4, BMPR1A, BRAF, BRCA1, BRCA2, BRD4, BRIP1, BTG1, BTK, BUB1B, C17orf39, CARD11, CARM1, CASP8, CAV1, CBFA2T3, CBFB, CBL, CCND1, CCND2, CCND3, CCNE1, CD274, CD276, CD40LG, CD44, CD79A, CD79B, CDC25A, CDC42, CDC73, CDH1, CDK12, CDK2, CDK4, CDK5, CDK6, CDK7, CDK8, CDK9, CDKN1A, CDKN1B, CDKN1C, CDKN2A, CDKN2B, CDKN2C, CDKN2D, CDX2, CEBPA, CEP57, CERK, CHEK1, CHEK2, CHN1, CHUK, CIC, CLTC, COL1A1, CRBN, CREBBP, CRKL, CRLF2, CSF1R, CSMD3, CSNK1G2, CTCF, CTLA4, CTNNA1, CTNNB1, CUL3, CUL4A, CUL4B, CYLD, CYP17A1, CYP19A1, CYP1B1, CYP2D6, DAXX, DCUN1D1, DDB2, DDIT3, DDR2, DGKB, DGKG, DGKI, DGKZ, DICER1, DIRAS3, DIS3, DIS3L2, DNMT1, DNMT3A, DNMT3B, DOT1L, DPYD, E2F1, E2F3, EED, EGF, EGFL7, EGFR, EIF1AX, ELOVL2, EMSY, ENPP2, EP300, EP400, EPCAM, EPHA2, EPHA3, EPHA5, EPHA8, EPHB1, EPHB2, EPHB4, EPHB6, EPO, ERBB2, ERBB3, ERBB4, ERCC1, ERCC2, ERCC3, ERCC4, ERCC5, ERCC6, ERG, ESR1, ESR2, ETS2, ETV1, ETV4, ETV6, EWSR1, EXT1, EXT2, EZH2, FAM123B (WTX), FAM175A, FAM46C, FANCA, FANCB, FANCC, FANCD2, FANCE, FANCF, FANCG, FANCI, FANCL, FANCM, FAS, FAT1, FAT3, FBXW7, FES, FGF10, FGF12, FGF14, FGF19, FGF23, FGF3, FGF4, FGF6, FGF7, FGFR1, FGFR2, FGFR3, FGFR4, FH, FHIT, FIGF, FLCN, FLNC, FLT1, FLT3, FLT4, FN1, FOS, FOXA1, FOXL2, FOXO1, FOXO3, FOXP1, FUBP1, FURIN, GAB1, GATA1, GATA2, GATA3, GMPS, GNA11, GNA13, GNAQ, GNAS, GPC3, GPR124, GRB2, GREM1, GRIN2A, GSK3B, GSTT1, H3F3C, HDAC1, HDAC2, HDAC3, HDAC4, HGF, HIF1A, HIST1H1C, HIST1H2BD, HIST1H3B, HLA-A, HMGA1, HNF1A, HOXA9, HOXD11, HRAS, HSP90AA1, ICAM1, ICOSLG, IDH1, IDH2, IFNG, IFNGR1, IGF1, IGF1R, IGF2, IGF2R, IGFBP3, IKBKE, IKZF1, IL10, IL2, IL2RA, IL7R, INHBA, INPP4A, INPP4B, INSR, IRF4, IRS1, IRS2, ITGB3, JAK1, JAK2, JAK3, JUN, KALRN, KAT2B, KDM5A, KDM5C, KDM6A, KDR, KEAP1, KIT, KLF4, KLF6, KLHL6, KRAS, LAMA1, LAMP1, LATS1, LATS2, LDHA, LMO1, LMO2, LRP1B, LTBP1, MAP2K1, MAP2K2, MAP2K4, MAP3K1, MAP3K13, MAPK1, MAPK3, MAPK9, MAX, MCL1, MDC1, MDM2, MDM4, MECOM, MED12, MEF2B, MEN1, MET, MINPP1, MITF, MLH1, MLL, MLL2, MLL3, MPL, MRE11, MRE11A, MSH2, MSH6, MST1R, MTOR, MUC1, MUTYH, MYC, MYCL1, MYCN, MYD88, MYH9, MYOD1, MYST3, MYST4, NAV3, NBN, NCOA2, NCOR1, NF1, NF2, NFE2L2, NFKBIA, NKX2-1, NKX3-1, NOS2, NOS3, NOTCH1, NOTCH2, NOTCH3, NOTCH4, NPM1, NR3C1, NRAS, NSD1, NTRK1, NTRK2, NTRK3, NUP214, NUP93, PAFAH1B2, PAK1, PAK3, PAK7, PALB2, PARK2, PARP1, PARP2, PARP3, PARP4, PAX5, PBRM1, PCNA, PDCD1, PDGFA, PDGFB, PDGFRA, PDGFRB, PDK1, PDPK1, PGR, PHOX2B, PIGS, PIK3C2G, PIK3C3, PIK3CA, PIK3CB, PIK3CD, PIK3CG, PIK3R1, PIK3R2, PIK3R3, PIM1, PLCB1, PLCG1, PLCG2, PLK2, PMAIP1, PML, PMS1, PMS2, PNRC1, POLE, PPARA, PPARG, PPARGC1A, PPP1R13L, PPP1R3A, PPP2CB, PPP2R1A, PPP2R1B, PPP2R2B, PRDM1, PRF1, PRKAR1A, PRKCA, PRKCG, PRKCZ, PRKDC, PRSS8, PTCH1, PTCH2, PTEN, PTGS2, PTK2, PTPN11, PTPRB, PTPRC, PTPRD, PTPRF, PTPRS, PTPRT, RAC1, RAD50, RAD51, RAD51B, RAD51C, RAD51D, RAD51L1, RAD52, RAD54L, RAF1, RARA, RASA1, R131, RBM10, RECQL4, REL, RET, RFWD2, RHBDF2, RHEB, RHOA, RICTOR, RIT1, RNF43, ROS1, RPA1, RPS6KA1, RPS6KA2, RPS6KA4, RPS6KB1, RPS6KB2, RPTOR, RUNX1, RUNX1T1, RYBP, SBDS, SDHA, SDHAF2, SDHB, SDHC, SDHD, SETD2, SF3B1, SH2B3, SH2D1A, SHC1, SHQ1, SKP2, SLX4, SMAD2, SMAD3, SMAD4, SMARCA4, SMARCB1, SMARCD1, SMO, SNCG, SOCS1, SOCS2, SOS1, SOX10, SOX17, SOX2, SOX9, SP1, SPEN, SPOP, SPRY2, SRC, STAG2, STAT4, STK11, STK40, SUFU, SUZ12, SYK, TALL TBX3, TCF12, TCF3, TEK, TERT, TET1, TET2, TFE3, TGFB3, TGFBR1, TGFBR2, THBS1, TIPARP, TK1, TLX1, TMEM127, TMPRSS2, TNFAIP3, TNFRSF14, TNK2, TOP1, TOP2A, TP53, TP63, TP73, TPM3, TPO, TPR, TRAF7, TRRAP, TSC1, TSC2, TSHR, U2AF1, UGT1A1, VDR, VEGFA, VHL, VTCN1, WISP3, WRN, WT1, XIAP, XPA, XPC, XPO1, XRCC3, YAP1, YES1, ZNF217, ZNF331, and ZNF703.

In some cases, a clinically actionable variant is a clinically actionable variant selected from Table 1.

TABLE 1
List of clinically actionable variants and therapeutic implications

| Variant | Amino Acid | | Chromosome | Protein | Variant | Therapeutic |
|---|---|---|---|---|---|---|
| Class | Location | Gene | Location | Location | Type | Implication |
| AKT | AKT1 E17 | AKT1 | | E17 | snv | sensitizing for AKT |
| activating | | | | | | or mTOR inhibitors |
| ALK | ALK C1156 | ALK | | C1156 | snv | sensitizing for ALK |
| activating | | | | | | inhibitors |
| ALK | ALK D1203 | ALK | | D1203 | snv | sensitizing for ALK |
| activating | | | | | | inhibitors |
| ALK | ALK F1174 | ALK | | F1174 | snv | sensitizing for ALK |
| activating | | | | | | inhibitors |
| ALK | ALK G1269 | ALK | | G1269 | snv | sensitizing for ALK |
| activating | | | | | | inhibitors |
| ALK | ALK L1152 | ALK | | L1152 | snv | sensitizing for ALK |
| activating | | | | | | inhibitors |
| ALK | ALK L1196 | ALK | | L1196 | snv | sensitizing for ALK |
| activating | | | | | | inhibitors |
| ALK | ALK L1198 | ALK | | L1198 | snv | sensitizing for ALK |
| activating | | | | | | inhibitors |
| ALK | ALK R1275 | ALK | | R1275 | snv | sensitizing for ALK |
| activating | | | | | | inhibitors |
| BRAF | BRAF D594 | BRAF | | D594 | snv | sensitizing for |
| activating | | | | | | BRAF inhibitors |
| BRAF | BRAF G466 | BRAF | | G466 | snv | sensitizing for |
| activating | | | | | | BRAF inhibitors |
| BRAF | BRAF G469 | BRAF | | G469 | snv | sensitizing for |
| activating | | | | | | BRAF inhibitors |
| BRAF | BRAF G596 | BRAF | | G596 | snv | sensitizing for |
| activating | | | | | | BRAF inhibitors |
| BRAF | BRAF L597 | BRAF | | L597 | snv | sensitizing for |
| activating | | | | | | BRAF inhibitors |
| BRAF | BRAF V600 | BRAF | | V600 | snv | sensitizing for |
| activating | | | | | | BRAF inhibitors |
| BRAF | BRAF K601 | BRAF | | K601 | snv | sensitizing for |
| activating | | | | | | BRAF inhibitors |
| BRAF | BRAF Y472 | BRAF | | Y472 | snv | sensitizing for |
| activating | | | | | | BRAF inhibitors |
| BRCA1 | BRCA1 | BRCA1 | | A1708 | snv | candidate for PARP |
| disabling | A1708 | | | | | inhibitors |
| BRCA1 | BRCA1 | BRCA1 | | C1787 | snv | candidate for PARP |
| disabling | C1787 | | | | | inhibitors |
| BRCA1 | BRCA1 C39 | BRCA1 | | C39 | snv | candidate for PARP |
| disabling | | | | | | inhibitors |
| BRCA1 | BRCA1 C44 | BRCA1 | | C44 | snv | candidate for PARP |
| disabling | | | | | | inhibitors |
| BRCA1 | BRCA1 C61 | BRCA1 | | C61 | snv | candidate for PARP |
| disabling | | | | | | inhibitors |
| BRCA1 | BRCA1 | BRCA1 | | G1706 | snv | candidate for PARP |
| disabling | G1706 | | | | | inhibitors |
| BRCA1 | BRCA1 | BRCA1 | | G1738 | snv | candidate for PARP |
| disabling | G1738 | | | | | inhibitors |
| BRCA1 | BRCA1 | BRCA1 | | G1788 | snv | candidate for PARP |
| disabling | G1788 | | | | | inhibitors |
| BRCA1 | BRCA1 I1766 | BRCA1 | | I1766 | snv | candidate for PARP |
| disabling | | | | | | inhibitors |
| BRCA1 | BRCA1 | BRCA1 | | L1764 | snv | candidate for PARP |
| disabling | L1764 | | | | | inhibitors |
| BRCA1 | BRCA1 L22 | BRCA1 | | L22 | snv | candidate for PARP |
| disabling | | | | | | inhibitors |
| BRCA1 | BRCA1 | BRCA1 | | M1775 | snv | candidate for PARP |
| disabling | M1775 | | | | | inhibitors |
| BRCA1 | BRCA1 | BRCA1 | | N1067 | snv | candidate for PARP |
| disabling | N1067 | | | | | inhibitors |
| BRCA1 | BRCA1 | BRCA1 | | R1495 | snv | candidate for PARP |
| disabling | R1495 | | | | | inhibitors |
| BRCA1 | BRCA1 | BRCA1 | | R1699 | snv | candidate for PARP |
| disabling | R1699 | | | | | inhibitors |
| BRCA1 | BRCA1 | BRCA1 | | S1715 | snv | candidate for PARP |
| disabling | S1715 | | | | | inhibitors |
| BRCA1 | BRCA1 | BRCA1 | | T1685 | snv | candidate for PARP |
| disabling | T1685 | | | | | inhibitors |
| BRCA1 | BRCA1 T37 | BRCA1 | | T37 | snv | candidate for PARP |
| disabling | | | | | | inhibitors |
| BRCA1 | BRCA1 | BRCA1 | | V1688 | del | candidate for PARP |
| disabling | V1688del | | | | | inhibitors |
| BRCA1 | BRCA1 | BRCA1 | | V1838 | snv | candidate for PARP |
| disabling | V1838 | | | | | inhibitors |
| BRCA2 | BRCA2 | BRCA2 | | D2723 | snv | candidate for PARP |
| disabling | D2723 | | | | | inhibitors |
| BRCA2 | BRCA2 | BRCA2 | | E2663 | snv | candidate for PARP |
| disabling | E2663 | | | | | inhibitors |
| BRCA2 | BRCA2 | BRCA2 | | G2748 | snv | candidate for PARP |
| disabling | G2748 | | | | | inhibitors |
| BRCA2 | BRCA2 I2627 | BRCA2 | | I2627 | snv | candidate for PARP |
| disabling | | | | | | inhibitors |
| BRCA2 | BRCA2 | BRCA2 | | L2653 | snv | candidate for PARP |
| disabling | L2653 | | | | | inhibitors |
| BRCA2 | BRCA2 | BRCA2 | | R2659 | snv | candidate for PARP |
| disabling | R2659 | | | | | inhibitors |
| BRCA2 | BRCA2 | BRCA2 | | R3052 | snv | candidate for PARP |
| disabling | R3052 | | | | | inhibitors |
| BRCA2 | BRCA2 | BRCA2 | | T2722 | snv | candidate for PARP |
| disabling | T2722 | | | | | inhibitors |
| BRCA2 | BRCA2 | BRCA2 | | W2626 | snv | candidate for PARP |
| disabling | W2626 | | | | | inhibitors |
| CDKN2A | CDKN2A | CDKN2A | | A73 | snv | candidate for CDK |
| disabling | A73 | | | | | 4/6 inhibitors |
| CDKN2A | CDKN2A C72 | CDKN2A | | C72 | snv | candidate for CDK |
| disabling | | | | | | 4/6 inhibitors |
| CDKN2A | CDKN2A M1 | CDKN2A | | M1 | snv | candidate for CDK |
| disabling | | | | | | 4/6 inhibitors |
| CDKN2A | CDKN2A | CDKN2A | | P114 | snv | candidate for CDK |
| disabling | P114 | | | | | 4/6 inhibitors |
| CDKN2A | CDKN2A R47 | CDKN2A | | R47 | snv | candidate for CDK |
| disabling | | | | | | 4/6 inhibitors |
| CDKN2A | CDKN2A R80 | CDKN2A | | R80 | snv | candidate for CDK |
| disabling | | | | | | 4/6 inhibitors |
| CDKN2A | CDKN2A | CDKN2A | | W110 | snv | candidate for CDK |
| disabling | W110 | | | | | 4/6 inhibitors |
| DDR2 | DDR2 S768 | DDR2 | | S768 | snv | candidate for CDK |
| activating | | | | | | 4/6 inhibitors |
| EGFR | EGFR | EGFR | Exon19 | A750 | del | sensitizing for |
| activating | A750del | | | | | EGFR inhibitors |
| EGFR | EGFR | EGFR | Exon19 | E746 | del | sensitizing for |
| activating | E746del | | | | | EGFR inhibitors |
| EGFR | EGFR | EGFR | Exon19 | E749 | del | sensitizing for |
| activating | E749del | | | | | EGFR inhibitors |
| EGFR | EGFR | EGFR | Exon19 | L747 | del | sensitizing for |
| activating | L747del | | | | | EGFR inhibitors |
| EGFR | EGFR | EGFR | Exon19 | P753 | del | sensitizing for |
| activating | P753del | | | | | EGFR inhibitors |
| EGFR | EGFR | EGFR | Exon19 | R748 | del | sensitizing for |
| activating | R748del | | | | | EGFR inhibitors |
| EGFR | EGFR | EGFR | Exon19 | S752 | del | sensitizing for |
| activating | S752del | | | | | EGFR inhibitors |
| EGFR | EGFR | EGFR | Exon19 | T751 | del | sensitizing for |
| activating | T751del | | | | | EGFR inhibitors |
| EGFR | EGFR | EGFR | Exon19 | A743 | ins | sensitizing for |
| activating | A743ins | | | | | EGFR inhibitors |
| EGFR | EGFR I740ins | EGFR | Exon19 | I740 | ins | sensitizing for |
| activating | | | | | | EGFR inhibitors |
| EGFR | EGFR I744ins | EGFR | Exon19 | I744 | ins | sensitizing for |
| activating | | | | | | EGFR inhibitors |
| EGFR | EGFR | EGFR | Exon19 | K739 | ins | sensitizing for |
| activating | K739ins | | | | | EGFR inhibitors |
| EGFR | EGFR | EGFR | Exon19 | P741 | ins | sensitizing for |
| activating | P741 ins | | | | | EGFR inhibitors |
| EGFR | EGFR | EGFR | Exon19 | V742 | ins | sensitizing for |
| activating | V742ins | | | | | EGFR inhibitors |
| EGFR | EGFR | EGFR | Exon20 | D770 | ins | sensitizing for |
| activating | D770ins | | | | | EGFR inhibitors |
| EGFR | EGFR | EGFR | Exon20 | H773 | ins | sensitizing for |
| activating | H773ins | | | | | EGFR inhibitors |
| EGFR | EGFR | EGFR | Exon20 | N771 | ins | sensitizing for |
| activating | N771ins | | | | | EGFR inhibitors |
| EGFR | EGFR | EGFR | Exon20 | P772 | ins | sensitizing for |
| activating | P772ins | | | | | EGFR inhibitors |
| EGFR | EGFR | EGFR | Exon20 | S768 | ins | sensitizing for |
| activating | S768ins | | | | | EGFR inhibitors |
| EGFR | EGFR | EGFR | Exon20 | V769 | ins | sensitizing for |
| activating | V769ins | | | | | EGFR inhibitors |
| EGFR | EGFR | EGFR | Exon20 | V774 | ins | sensitizing for |
| activating | V774ins | | | | | EGFR inhibitors |
| EGFR | EGFR E709 | EGFR | | E709 | snv | sensitizing for |
| activating | | | | | | EGFR inhibitors |
| EGFR | EGFR G719 | EGFR | | G719 | snv | sensitizing for |
| activating | | | | | | EGFR inhibitors |
| EGFR | EGFR L858 | EGFR | | L858 | snv | sensitizing for |
| activating | | | | | | EGFR inhibitors |
| EGFR | EGFR L861 | EGFR | | L861 | snv | sensitizing for |
| activating | | | | | | EGFR inhibitors |
| EGFR | EGFR T790 | EGFR | | T790 | snv | sensitizing for |
| activating | | | | | | EGFR inhibitors |
| EGFR | EGFR | EGFR | | A763 | ins | sensitizing for |
| activating | A763ins | | | | | EGFR inhibitors |
| FLT3 | FLT3 D835 | FLT3 | | D835 | snv | sensitizing for |
| activating | | | | | | FLT3 inhibitors |
| FLT3 | FLT3 F691 | FLT3 | | F691 | snv | sensitizing for |
| activating | | | | | | FLT3 inhibitors |
| FLT3 | FLT3 N841 | FLT3 | | N841 | snv | sensitizing for |
| activating | | | | | | FLT3 inhibitors |
| FLT3 | FLT3 Y842 | FLT3 | | Y842 | snv | sensitizing for |
| activating | | | | | | FLT3 inhibitors |
| GNAQ | GNAQ Q209 | GNAQ | | Q209 | snv | sensitizing for |
| activating | | | | | | FLT3 inhibitors |
| KIT activating | KIT 554del | KIT | | 554del | del | sensitizing for KIT |
| | | | | | | inhibitors |
| KIT activating | KIT 556ins | KIT | | 556ins | ins | sensitizing for KIT |
| | | | | | | inhibitors |
| KIT activating | KIT 566del | KIT | | 566del | del | sensitizing for KIT |
| | | | | | | inhibitors |
| KIT activating | KIT 575ins | KIT | | 575ins | ins | sensitizing for KIT |
| | | | | | | inhibitors |
| KIT activating | KIT 579del | KIT | | 579del | del | sensitizing for KIT |
| | | | | | | inhibitors |
| KIT activating | KIT A829 | KIT | | A829 | snv | sensitizing for KIT |
| | | | | | | inhibitors |
| KIT activating | KIT D816 | KIT | | D816 | snv | sensitizing for KIT |
| | | | | | | inhibitors |
| KIT activating | KIT D820 | KIT | | D820 | snv | sensitizing for KIT |
| | | | | | | inhibitors |
| KIT activating | KIT E583ins | KIT | | E583ins | ins | sensitizing for KIT |
| | | | | | | inhibitors |
| KIT activating | KIT K550 | KIT | | K550N | snv | sensitizing for KIT |
| | | | | | | inhibitors |
| KIT activating | KIT K558 | KIT | | K558 | snv | sensitizing for KIT |
| | | | | | | inhibitors |
| KIT activating | KIT K642 | KIT | | K642 | snv | sensitizing for KIT |
| | | | | | | inhibitors |
| KIT activating | KIT L576 | KIT | | L576 | snv | sensitizing for KIT |
| | | | | | | inhibitors |
| KIT activating | KIT N822 | KIT | | N822 | snv | sensitizing for KIT |
| | | | | | | inhibitors |
| KIT activating | KIT V559 | KIT | | V559 | snv | sensitizing for KIT |
| | | | | | | inhibitors |
| KIT activating | KIT V559del | KIT | | V559 | del | sensitizing for KIT |
| | | | | | | inhibitors |
| KIT activating | KIT V560 | KIT | | V560 | snv | sensitizing for KIT |
| | | | | | | inhibitors |
| KIT activating | KIT V654 | KIT | | V654 | snv | sensitizing for KIT |
| | | | | | | inhibitors |
| KIT activating | KIT W557 | KIT | | W557 | snv | sensitizing for KIT |
| | | | | | | inhibitors |
| KIT activating | KIT Y553 | KIT | | Y553 | snv | sensitizing for KIT |
| | | | | | | inhibitors |
| KIT activating | KIT Y823 | KIT | | Y823 | snv | sensitizing for KIT |
| | | | | | | inhibitors |
| KRAS | KRAS A146 | KRAS | | A146 | snv | sensitizing for |
| activating | | | | | | MEK inhibitors |
| KRAS | KRAS G12 | KRAS | | G12 | snv | sensitizing for |
| activating | | | | | | MEK inhibitors |
| KRAS | KRAS G13 | KRAS | | G13 | snv | sensitizing for |
| activating | | | | | | MEK inhibitors |
| KRAS | KRAS K117 | KRAS | | K117 | snv | sensitizing for |
| activating | | | | | | MEK inhibitors |
| KRAS | KRAS Q61 | KRAS | | Q61 | snv | sensitizing for |
| activating | | | | | | MEK inhibitors |
| MAP2K1 | MAP2K1 | MAP2K1 | | C121 | snv | candidate for MEK |
| activating | C121 | | | | | inhibitors |
| MAP2K1 | MAP2K1 D67 | MAP2K1 | | D67 | snv | candidate for MEK |
| activating | | | | | | inhibitors |
| MAP2K1 | MAP2K1 K57 | MAP2K1 | | K57 | snv | candidate for MEK |
| activating | | | | | | inhibitors |
| MAP2K1 | MAP2K1 Q56 | MAP2K1 | | Q56 | snv | candidate for MEK |
| activating | | | | | | inhibitors |
| Exceptional | MTOR E2014 | MTOR | | E2014 | snv | exceptional |
| Response | | | | | | response to |
| | | | | | | everolimus |
| Exceptional | MTOR E2419 | MTOR | | E2419 | snv | exceptional |
| Response | | | | | | response to |
| | | | | | | everolimus |
| NRAS | NRAS G12 | NRAS | | G12 | snv | candidate for MEK |
| activating | | | | | | inhibitors |
| NRAS | NRAS Q61 | NRAS | | Q61 | snv | candidate for MEK |
| activating | | | | | | inhibitors |
| PIK3CA | PIK3CA D549 | PIK3CA | | D549 | snv | candidate for PI3K |
| activating | | | | | | or AKT or mTOR |
| | | | | | | inhibitors |
| PIK3CA | PIK3CA E542 | PIK3CA | | E542 | snv | candidate for PI3K |
| activating | | | | | | or AKT or mTOR |
| | | | | | | inhibitors |
| PIK3CA | PIK3CA E545 | PIK3CA | | E545 | snv | candidate for PI3K |
| activating | | | | | | or AKT or mTOR |
| | | | | | | inhibitors |
| PIK3CA | PIK3CA | PIK3CA | | H1047 | snv | candidate for PI3K |
| activating | H1047 | | | | | or AKT or mTOR |
| | | | | | | inhibitors |
| PIK3CA | PIK3CA Q546 | PIK3CA | | Q546 | snv | candidate for PI3K |
| activating | | | | | | or AKT or mTOR |
| | | | | | | inhibitors |
| PIK3R1 | PIK3R1 E160 | PIK3R1 | | E160 | snv | candidate for PI3K |
| disabling | | | | | | or AKT or mTOR |
| | | | | | | inhibitors |
| PIK3R1 | PIK3R1 | PIK3R1 | | L370 | del | candidate for PI3K |
| disabling | L370del | | | | | or AKT or mTOR |
| | | | | | | inhibitors |
| PIK3R1 | PIK3R1 R348 | PIK3R1 | | R348 | snv | candidate for PI3K |
| disabling | | | | | | or AKT or mTOR |
| | | | | | | inhibitors |
| PIK3R1 | PIK3R1 R358 | PIK3R1 | | R358 | snv | candidate for PI3K |
| disabling | | | | | | or AKT or mTOR |
| | | | | | | inhibitors |
| PTCH1 | PTCH1 | PTCH1 | | G1093 | snv | candidate for SMO |
| disabling | G1093 | | | | | inhibitors |
| PTCH1 | PTCH1 G238 | PTCH1 | | G238 | snv | candidate for SMO |
| disabling | | | | | | inhibitors |
| PTCH1 | PTCH1 P1198 | PTCH1 | | P1198 | snv | candidate for SMO |
| disabling | | | | | | inhibitors |
| PTCH1 | PTCH1 P644 | PTCH1 | | P644 | snv | candidate for SMO |
| disabling | | | | | | inhibitors |
| PTCH1 | PTCH1 K838 | PTCH1 | | K838 | snv | candidate for SMO |
| disabling | | | | | | inhibitors |
| PTCH1 | PTCH1 S683 | PTCH1 | | S683 | snv | candidate for SMO |
| disabling | | | | | | inhibitors |
| PTCH1 | PTCH1 T1195 | PTCH1 | | T1195 | snv | candidate for SMO |
| disabling | | | | | | inhibitors |
| PTCH1 | PTCH1 W236 | PTCH1 | | W236 | snv | candidate for SMO |
| disabling | | | | | | inhibitors |
| PTCH1 | PTCH1 W844 | PTCH1 | | W844 | snv | candidate for SMO |
| disabling | | | | | | inhibitors |
| PTCH1 | PTCH1 W863 | PTCH1 | | W863 | snv | candidate for SMO |
| disabling | | | | | | inhibitors |
| PTEN | PTEN | PTEN | | K267 | del | candidate for |
| disabling | K267del | | | | | p110beta or AKT |
| | | | | | | or mTOR inhibitors |
| PTEN | PTEN R159 | PTEN | | R159 | snv | candidate for |
| disabling | | | | | | p110beta or AKT |
| | | | | | | or mTOR inhibitors |
| PTEN | PTEN R233 | PTEN | | R233 | snv | candidate for |
| disabling | | | | | | p110beta or AKT |
| | | | | | | or mTOR inhibitors |
| PTEN | PTEN A126 | PTEN | | A126 | snv | candidate for |
| disabling | | | | | | p110beta or AKT |
| | | | | | | or mTOR inhibitors |
| PTEN | PTEN C124 | PTEN | | C124 | snv | candidate for |
| disabling | | | | | | p110beta or AKT |
| | | | | | | or mTOR inhibitors |
| PTEN | PTEN D162 | PTEN | | D162 | snv | candidate for |
| disabling | | | | | | p110beta or AKT |
| | | | | | | or mTOR inhibitors |
| PTEN | PTEN D92 | PTEN | | D92 | snv | candidate for |
| disabling | | | | | | p110beta or AKT |
| | | | | | | or mTOR inhibitors |
| PTEN | PTEN G127 | PTEN | | G127 | snv | candidate for |
| disabling | | | | | | p110beta or AKT |
| | | | | | | or mTOR inhibitors |
| PTEN | PTEN G129 | PTEN | | G129 | snv | candidate for |
| disabling | | | | | | p110beta or AKT |
| | | | | | | or mTOR inhibitors |
| PTEN | PTEN H123 | PTEN | | H123 | snv | candidate for |
| disabling | | | | | | p110beta or AKT |
| | | | | | | or mTOR inhibitors |
| PTEN | PTEN H93 | PTEN | | H93 | snv | candidate for |
| disabling | | | | | | p110beta or AKT |
| | | | | | | or mTOR inhibitors |
| PTEN | PTEN K125 | PTEN | | K125 | snv | candidate for |
| disabling | | | | | | p110beta or AKT |
| | | | | | | or mTOR inhibitors |
| PTEN | PTEN K128 | PTEN | | K128 | snv | candidate for |
| disabling | | | | | | p110beta or AKT |
| | | | | | | or mTOR inhibitors |
| PTEN | PTEN Q171 | PTEN | | Q171 | snv | candidate for |
| disabling | | | | | | p110beta or AKT |
| | | | | | | or mTOR inhibitors |
| PTEN | PTEN R130 | PTEN | | R130 | snv | candidate for |
| disabling | | | | | | p110beta or AKT |
| | | | | | | or mTOR inhibitors |
| PTEN | PTEN R173 | PTEN | | R173 | snv | candidate for |
| disabling | | | | | | p110beta or AKT |
| | | | | | | or mTOR inhibitors |
| PTEN | PTEN V166 | PTEN | | V166 | snv | candidate for |
| disabling | | | | | | p110beta or AKT |
| | | | | | | or mTOR inhibitors |

### Quality of Data/Quality Score

The methods and systems described herein provide for calculating one or more quality score. The methods and systems described herein further provide for assigning one or more quality score to a subset of sequencing data. One or more quality score may comprise a read depth (or depth of coverage), a mapping quality, or a base call quality.

In one case, a read depth or depth of coverage is determined for a genomic region comprising the genetic variant. “Read depth” and “depth of coverage” are used herein interchangeably and refer to the average number of times a nucleotide base is “called” in a sequencing reaction. Generally, a higher read depth provides greater accuracy with which any given nucleotide base can be called. For example, a read depth of 10× means that any given nucleotide will be called on average ten times. It should be understood that read depth may not be uniform. For example, certain regions of the genome may be more challenging to sequence accurately for e.g., regions with high GC content. In other examples, sequencing bias can create a lack of uniformity in sequencing data. Sequencing bias may be random or non-random. In some cases, a regional read depth is determined for a genomic region. In some cases, the methods may comprise determining a read depth for one or more genomic regions of interest. A predetermined threshold may be selected such that genetic variants identified within a genomic region of interest with a quality score greater than the predetermined threshold is “called” with a level of confidence, and genetic variants identified within sequencing data with a quality score less than the predetermined threshold are not “called” with a level of confidence. In one example, a genetic variant may be identified in a genomic region with a sequencing read depth of 50×. In this example, the read depth may be sufficient to “call” the genetic variant with a level of confidence. In another example, a genetic variant may be identified in a genomic region with a sequencing read depth of 5×. In this example, the read depth may not be sufficient to “call” the genetic variant with a level of confidence. A read depth may include, without limitation, 1×, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 60×, 70×, 80×, 90×, 100×, 200×, 300×, 400×, 500×, 600×, 700×, 800×, 900×, 1000×, or greater.

In some cases, the quality score is comprised of a base call quality score. The base call quality score may be a Phred quality score. The Phred quality score may be assigned to each base call in automated sequencer traces and may be used to compare the efficacy of different sequencing methods. The Phred quality score (Q) may be defined as a property which is logarithmically related to the base-calling error probabilities (P). The Phred quality score (Q) may be calculated as Q=−10 log₁₀ P. The Phred quality score of the one or more sequencing reactions may be similar to the Phred quality score of current sequencing methods. The Phred quality score of the one or more sequencing methods may be within 1, 2, 3, 4, 5, 6, 7, 8, 9, 10 of the Phred quality score of the current sequencing methods. The Phred quality score of the one or more sequencing methods may be less than the Phred quality score of the one or more sequencing methods. The Phred quality score of the one or more sequencing methods may be at least about 10, 9, 8, 7, 6, 5, 4, 3, 2, 1 less than the Phred quality score of the one or more sequencing methods. The Phred quality score of the one or more sequencing methods may be greater than 5, 6, 7, 8, 9, 10, 11, 12, 13, 14, 15, 16, 17, 18, 19, 20, 25, or 30. The Phred quality score of the one or more sequencing methods may be greater than 35, 40, 41, 42, 43, 44, 45, 46, 47, 48, 49, 50, 51, 52, 53, 54, 55, 56, 57, 58, 59, or 60. The Phred quality score of the one or more sequencing methods may be at least 35, 36, 37, 38, 39, 40, 41, 42, 43, 44, 45, 46, 47, 48, 49, 50, 51, 52, 53, 54, 55, 56, 57, 58, 59, 60 or more.

In some cases, the quality score is comprised of a mapping quality score. The mapping quality score may indicate the accuracy with which a sequence has been mapped or aligned to a reference sequence. Mapping quality (Qm) scores can be calculated for each aligned read in several different ways. In one particular example, the aligner will provide a mapping quality score (MQS) in which:

$$MQS = \{ \begin{matrix}( {Σ}_{i ∈ bm} ( 1 - {p}_{i} ) - {Σ}_{i ∈ bmn} ( 1 - {p}_{i} ) ) \times 60 \text{/} L , if uniquely mapped \\ 0 , if mapped to > 1 best location\end{matrix}$$

wherein L is the read length, pₜ is the base-calling p-value for the ith base in the read, bₘ is the set of locations of matched bases, and bₘₘ is the set of locations of mismatched bases. Base-calling p-values are computed from base quality score, transformed from the Phred scale. The mapping quality score may be in a range from 0-60. In some cases, the mapping quality score of the one or more sequencing methods is at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60.

In some cases, the quality scores can be assigned a confidence score using empirical, machine learning methods. In a particular example, the quality score is based upon 4 values; the total read depth at the specific variant location, the proportion of reads containing the variant, the mean quality of the non-variant base calls at the location and the difference in mean quality for the variant base calls. Using a large collection of samples with known variants processed in a plurality of laboratories and utilizing a plurality of processing methods, a model is trained that associates the state of the input quality variables to the expected likelihood of a correct variant call (positive and negative treated similarly). The model derived in this way defines an n-dimensional response surface, with n=the number of input variables, trained on all variants taken together to provide the statistical power needed to construct a response surface over the full range of inputs. The response surface is stored in the form of equations to be used by a Quality Scoring Algorithm to assign a confidence score between 1 and 100% to the absence or presence call for each variant in the test panel, for an individual patient sample processed and reported.

### Samples

A subject can provide a biological sample for genetic screening. The biological sample can be any substance that is produced by the subject. Generally, the biological sample is any tissue taken from the subject or any substance produced by the subject. Non-limiting examples of biological samples can include blood, plasma, saliva, cerebrospinal fluid (CSF), cheek tissue (i.e., from a cheek swab), urine, feces, skin, hair, organ tissue, and the like. In some cases, the biological sample is a solid tumor or a biopsy of a solid tumor. In some cases, the biological sample is a formalin-fixed, paraffin-embedded (FFPE) tissue sample. The biological sample can be any biological sample that comprises nucleic acids. The term “nucleic acid” as used herein generally refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. The nucleic acid molecules can be DNA or RNA, or any combination thereof. RNA can comprise mRNA, miRNA, piRNA, siRNA, tRNA, rRNA, sncRNA, snoRNA and the like. DNA can comprise cDNA, genomic DNA, mitochondrial DNA, exosomal DNA, viral DNA and the like. In particular cases, the DNA is genomic DNA. Nucleic acids can be isolated from biological cells or can be cell-free nucleic acids (i.e., circulating DNA). In particular examples, the DNA is tumor DNA. In other particular examples, the RNA is tumor RNA. In some cases, the DNA is fetal DNA.

Biological samples may be derived from a subject. The subject may be a mammal, a reptile, an amphibian, an avian, or a fish. The mammal may be a human, ape, orangutan, monkey, chimpanzee, cow, pig, horse, rodent, bird, reptile, dog, cat, or other animal. A reptile may be a lizard, snake, alligator, turtle, crocodile, and tortoise. An amphibian may be a toad, frog, newt, and salamander. Examples of avians include, but are not limited to, ducks, geese, penguins, ostriches, and owls. Examples of fish include, but are not limited to, catfish, eels, sharks, and swordfish. Preferably, the subject is a human. The subject may suffer from a disease or condition.

### Diseases

The methods and systems disclosed herein may be particularly suited for diagnosing a disease. In some cases, the methods and systems disclosed herein may be utilized to identify clinically actionable variants known to alter or affect the efficacy of a therapeutic regimen for treating a disease. In some cases, the disease is cancer. Non-limiting examples of cancers can include: Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor.

In some cases, the methods and systems disclosed herein may be utilized to identify clinically actionable variants known to alter or affect the efficacy of a therapeutic regimen for treating a disease. In some cases, the disease is an infectious disease, including bacteria, virus, fungal, or protozoan where the methods and systems could aid in identifying the primary pathogen(s), or assess variants that may increase risk of treatment, adverse effects and/or immune system response.

In some cases, the disease is a neurodegenerative disease, including, without limitation, Alzheimers, Dementia, Parkinsons and others, wherein the methods and systems may be used to identify treatable subtypes and match them to drugs now in development and identify pharmacogenetic variants that could influence dosing. In some cases, the disease is a neurological disorder, including, without limitation, intellectual development delay, epilepsy, or autism.

In some cases, the disease is an addiction disorder, wherein the methods and systems may identify subtypes based upon variants in receptor-signaling genes, and endorphin, dopamine or related pleasure seeking pathways that may be treatable.

In some cases the disease is an endocrine disease. Non-limiting examples include Acromegaly, Addison's Disease, Adrenal Disorders, Cushing's Syndrome, De Quervain's Thyroiditis, Diabetes, Gestational Diabetes, Goiters, Graves' Disease, Growth Disorders, Growth Hormone Deficiency, Hashimoto's Thyroiditis, Hyperglycemia, Hyperparathyroidism, Hyperthyroidism, Hypoglycemia, Hypoparathyroidism, Hypothyroidism, Low Testosterone, Multiple Endocrine Neoplasia Type 1, Type 2A, Type 2B, Obesity, Osteoporosis, Parathyroid Diseases, Pheochromocytoma, Pituitary Disorders, Pituitary Tumors, Polycystic Ovary Syndrome, Prediabetes, Silent Thyroiditis, Thyroid Diseases, Thyroid Nodules, Thyroiditis, Turner Syndrome, Type 1 Diabetes, and Type 2 Diabetes.

In some cases, the disease is an autoimmune disease. Non-limiting examples include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome**, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, Wegener's granulomatosis (now termed Granulomatosis with Polyangiitis (GPA).

In some cases, the disease is a cardiovascular disease, wherein the methods and systems can be used to identify variants that are associated with improved response to treatments currently available and those in development for use in the clinical setting to better match the individual patient to treatments.

### Biomedical Reports

The methods and systems disclosed herein provide for one or more biomedical reports. Examples of reports that can be generated by the methods and systems of the disclosure are depicted in FIGS. 2-5. The results of methods described herein may be presented on one or more biomedical reports. The one or more biomedical reports may be generated or produced by the systems of the disclosure. The one or more biomedical reports may be provided as a printed or electronic format to an end user (i.e., a healthcare provider or a patient). The biomedical report may provide a plurality of reporting factors. The biomedical report can provide a list of classified genetic variants. Genetic variants may be classified as absent, present, or indeterminate according to the methods disclosed herein. The specific genetic variant tested may be identified in the biomedical report (e.g., G12A) as well as the corresponding gene name (e.g., KRAS). The biomedical report may further provide the classification of the specific genetic variant (e.g., “present”). The biomedical report may provide the type of variant (e.g., activating mutation). The biomedical report may provide a data quality score for each variant tested. The data quality score may be the read depth, base call quality, mapping quality, or a combination thereof. In particular examples, the biomedical report provides the read depth for each variant tested. In some cases, the biomedical report can provide a treatment plan or recommendation based on the classification of a clinically actionable variant. For example, a biomedical report may identify the presence of an activating mutation in the KRAS gene and recommend that the patient be treated with a therapy indicated for cancers with known KRAS mutations (e.g., a MEK inhibitor). In some cases, the patient may be currently receiving treatment and the biomedical report may indicate that the patient should halt treatment or start a different treatment (e.g., the presence of a variant indicates a second therapy is more effective than the first therapy).

### Systems of the Disclosure

The disclosure further provides computer-based systems for performing the methods described herein. In some aspects, the systems can be utilized for determining and reporting the presence or absence of genetic variants in a sample. The system can comprise one or more client components. The one or more client components can comprise a user interface. The system can comprise one or more server components. The server components can comprise one or more memory locations. The one or more memory locations can be configured to receive a data input. The data input can comprise sequencing data. The sequencing data can be generated from a nucleic acid sample from a subject. Non-limiting examples of sequencing data suitable for use with the systems of this disclosure have been described. The system can further comprise one or more computer processor. The one or more computer processor can be operably coupled to the one or more memory locations. The one or more computer processor can be programmed to map the sequencing data to a reference sequence. The one or more computer processor can be further programmed to determine a presence or absence of a genetic variant from the sequencing data. The determining step can comprise any of the methods described herein. The determining can comprise assigning a quality score to a genomic region comprising the genetic variant to generate a classified genetic variant based on the quality score. The genetic variant can be a clinically actionable variant. In some cases, the clinically actionable variant can be classified as present if the clinically actionable variant is determined to be present and the quality score is greater than a predetermined threshold. In some cases, the clinically actionable variant can be classified as absent if the clinically actionable variant is determined to be absent and the quality score is greater than a predetermined threshold. In some cases, the clinically actionable variant is classified as indeterminate if the quality score is less than a predetermined threshold. The one or more computer processor can be further programmed to generate an output for display on a screen. The output can comprise one or more reports identifying the classified genetic variant.

The systems described herein can comprise one or more client components. The one or more client components can comprise one or more software components, one or more hardware components, or a combination thereof. The one or more client components can access one or more services through one or more server components. The one or more services can be accessed by the one or more client components through a network. “Services” is used herein to refer to any product, method, function, or use of the system. For example, a user can place an order for a genetic test. The order can be placed through the one or more client components of the system and the request can be transmitted through a network to the one or more server components of the system. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The systems can comprise one or more memory locations (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus, such as a motherboard. The storage unit can be a data storage unit (or data repository) for storing data. In one example, the one or more memory locations can store the received sequencing data.

The systems can comprise one or more computer processors. The one or more computer processors may be operably coupled to the one or more memory locations to e.g., access the stored sequencing data. The one or more computer processors can implement machine executable code to carry out the methods described herein. For instance, the one or more computer processors can execute machine readable code to map a sequencing data input to a reference sequence or to assign a quality score to a genomic region comprising a genetic variant.

The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, can be compiled during runtime, or can be interpreted during runtime. The code can be supplied in a programming language that can be selected to enable the code to execute in a pre-compiled, as-compiled or interpreted fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The systems disclosed herein can include or be in communication with one or more electronic displays. The electronic display can be part of the computer system, or coupled to the computer system directly or through the network. The computer system can include a user interface (UI) for providing various features and functionalities disclosed herein. Examples of UIs include, without limitation, graphical user interfaces (GUIs) and web-based user interfaces. The UI can provide an interactive tool by which a user can utilize the methods and systems described herein. By way of example, a UI as envisioned herein can be a web-based tool by which a healthcare practitioner can order a genetic test, customize a list of genetic variants to be tested, and receive and view a biomedical report.

The methods disclosed herein may comprise biomedical databases, genomic databases, biomedical reports, disease reports, case-control analysis, and rare variant discovery analysis based on data and/or information from one or more databases, one or more assays, one or more data or results, one or more outputs based on or derived from one or more assays, one or more outputs based on or derived from one or more data or results, or a combination thereof.

### Machine Executable Code

As described herein, one or more computer processors can implement machine executable code to perform the methods of the disclosure. Machine executable code can comprise any number of open-source or closed-source software. The machine executable code can be implemented to analyze a data input. The data input can be sequencing data generated from one or more sequencing reactions. The computer process can be operably coupled to at least one memory location. The computer processor can access the sequencing data from the at least one memory location. In some cases, the computer processor can implement machine executable code to map the sequencing data to a reference sequence. In some cases, the computer processor can implement machine executable code to determine a presence or absence of a genetic variant from the sequencing data. The genetic variant can be e.g., a clinically actionable variant. In some cases, the computer processor can implement machine executable code to calculate a quality score for at least one genomic region comprising a genetic variant. In some cases, the computer processor can implement machine executable code to assign a quality score to at least one genomic region comprising a genetic variant. In some cases, the computer processor can implement machine executable code to classify a genetic variant based on the assigned quality score. In some cases, the computer processor can implement machine executable code to generate an output for display on a screen (e.g., a biomedical report) identifying the classified genetic variant.

Machine executable code (or machine readable code) can include one or more sequence alignment software. Sequence alignment software can include DNA-seq aligners. Non-limiting examples of DNA-seq aligners suitable to perform the methods of the disclosure include BLAST, CS-BLAST, CUDASW++, FASTA, GGSEARCH/GLSEARCH, HMMER, HHpred/HHsearch, IDF, Infernal, KLAST, PSI-BLAST, PSI-Search, ScalaBLAST, Sequilab, SAM, SSEARCH, SWAPHI, SWAPHI-LS, SWIPE, ACANA, AlignMe, Bioconductor, Biostrings::pairwiseAlignment, BioPerl dpAlign, BLASTZ, LASTZ, CUDAlign, DNADot, DOTLET, FEAST, G-PAS, GapMis, JAligner, K*Sync, LALIGN, NW-align, mAlign, matcher, MCALIGN2, MUMmer, needle, Ngila, Path, PatternHunter, ProbA (propA), PyMOL, REPuter, SABERTOOTH, Satsuma, SEQALN, SIM, GAP, LAP, NAP, SPA, Sequences Studio, SWIFT Suit, stretcher, tranalign, UGENE, water, wordmatch, YASS, ABA, ALE, AMAP, anon., BAli-Phy, Base-By-Base, CHAOS/DIALIGN, ClustalW, CodonCode Aligner, Compass, DECIPHER, DIALIGN-TX, DIALIGN-T, DNA Alignment, DNA Baser Sequence Assembler, EDNA, FSA, Geneious, KAlign, MAFFT, MARNA, MAVID, MSA, MSAProbes, MULTALIN, Multi-LAGAN, MUSCLE, Opal, Pecan, Phylo, Praline, PicXAA, POA, Probalign, ProbCons, PROMALS3D, PRRN/PRRD, PSAlign, RevTrans, SAGA, Se-A1, StatAlign, Stemloc, T-Coffee, UGENE, VectorFriends, GLProbs, ACT, AVID, BLAT, GMAP, Splign, Mauve, MGA, Mulan, Multiz, PLAST-ncRNA, Sequerome, Sequilab, Shuffle-LAGAN, SIBSim4, SLAM, BarraCUDA, BBMap, BFAST, BLASTN, Bowtie, HIVE-Hexagon, BWA, BWA-MEM, BWA-PSSM, CASHX, Cloudburst, CUDA-EC, CUSHAW, CUSHAW2, CUSHAW2-GPU, CUSHAW3, drFAST, ELAND, ERNE, GASSST, GEM, Genalice MAP, Geneious Assembler, GensearchNGS, GMAP, GSNAP, GNUMAP, iSSAC, LAST, MAQ, mrFAST, mrsFAST, MOM, MOSAIK, MPscan, Novoalign, NovoalignCS, NextGENe, NextGenMap, Omixon, PALMapper, Partek, PASS, PerM, PRIMEX, QPalma, RazerS, REAL, cREAL, RMAP, rNA, RTG Investigator, Segemehl, SeqMap, Shrec, SHRIMP, SLIDER, SOAP, SOAP2, SOAP3, SOAP3-dp, SOCS, SSAHA, SSAHA2, Stampy, SToRM, Subread, Subjunc, Taipan, VelociMapper, XPressAlign, ZOOM, and YAHA. In some cases, sequence alignment software can include RNA-seq aligners. Non-limiting examples of RNA-seq aligners suitable to perform the methods of the disclosure include Bowtie, Cufflinks, Erange, GMAP, GSNAP, GSTRUCT, GEM, IsoformEx, HISAT, HPG aligner, HMMSplicer, MapAL, MapSplice, Olego, OSA, PALMapper, PASS, RNA MATE, ReadsMap, RUM, RNASEQR, SAMMate, SOAPSplice, SMALT, STAR1, STAR2, SpliceSeq, SpliceMap, Subread, Subjunc, TopHat1, TopHat2, and X-Mate.

Machine executable code can include one or more alignment visualization software. Alignment visualization software can include, without limitation, Ale, IVistMSA, AliView, Base-By-Base, BioEdit, BioNumerics, BoxShade, CINEMA, CLC viewer, ClustalX viewer, Cylindrical BLAST viewer, DECIPHER, Discovery Studio, DnaSP, emacs-biomode, Genedoc, Geneious, Integrated Genome Browser (IGB), Integrative Genomics Viewer (IGV), Jalview 2, JEvTrace, JSAV, Maestro, MEGA, Multiseq, MView, PFAAT, Ralee, S2S RNA editor, Seaview, Sequilab, SeqPop, Sequlator, SnipViz, Strap, Tablet, UGENE, VISSA sequence/structure viewer, Artemis, Savant, DNApy, Alignment Annotator, Google Genomics API Browser, and PyBamView.

Machine executable code can include one or more variant calling software. Variant calling software can include germline or somatic callers which identify all single nucleotide variants, insertions and deletions and report read counts supporting the presence of the identified variants. Examples of germline or somatic callers can include, without limitation, CRISP, SNVer, Platypus, BreaKmer, Gustaf, GATK, VarScan, VarScan2, Somatic Sniper and SAMTools. Variant calling software can include CNV identifiers, which identify copy number changes. Examples of CNV identifiers can include, without limitation, CNVnator, RDXplorer, CONTRA, and ExomeCNV. Variant calling software can include structural variant identifiers, which identify larger insertions, deletions, inversions, inter- and intra-chromosomal translocations in DNA-seq data, or fusion products in RNA-seq data. Examples of structural variant identifiers can include, without limitation, BreakDancer, Breakpointer, ChimeraScan, DeFuse, Delly, CLEVER, EBARDenovo, FusionAnalyser, FusionCatcher, FusionHunter, FusionMap, Fusion Seq, GASBPro, JAFFA, PRADA, SOAPFuse, SOAPfusion, SVMerge, and TopHat-Fusion.

Machine executable code may comprise one or more algorithms. The one or more algorithms may be used to implement the methods of the disclosure. One or more algorithm can comprise a feature counting algorithm. The feature counting algorithm can be utilized to compute the maximum, minimum or average read depth within each region of a given region list. The output of the feature counting algorithm may be utilized to compute the certainty in the absence of the variant and to confirm the certainty in the presence of the variant. One or more algorithm can comprise a reference builder algorithm. The reference builder algorithm can convert the variants selected by the user for the inclusion in the test panel into chromosomal locations (i.e., a genetic address). One or more algorithm can comprise a quality scoring algorithm. The quality scoring algorithm can assign a confidence score between 1 and 100% to the absence or presence call for each variant based on quality inputs. One or more algorithm can comprise a direct mining algorithm. The direct mining algorithm can utilize a reference sequence in the vicinity of the variant on the test panel to query the raw read data and assemble the evidence to support the presence or absence of the variant.

### Computer Systems

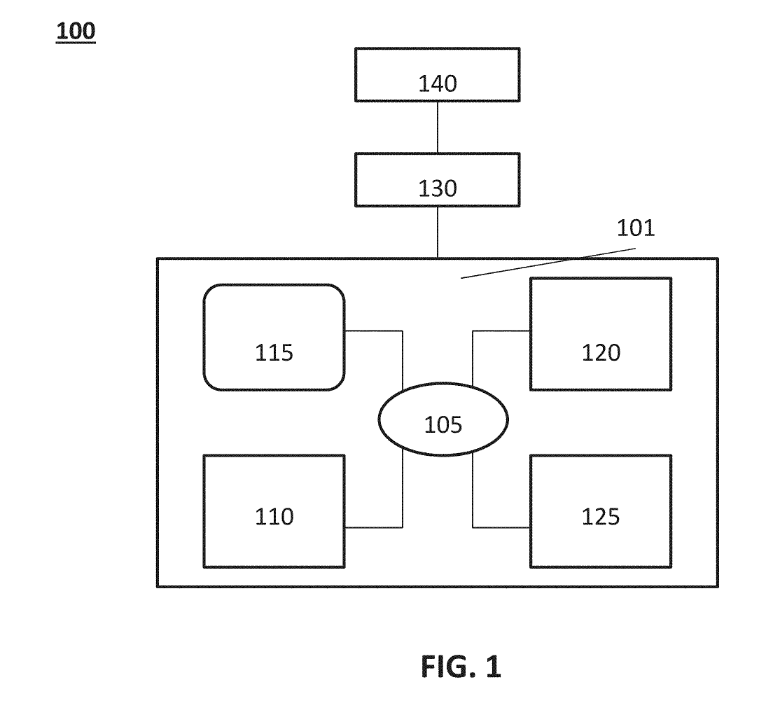

The systems of the disclosure may comprise one or more computer systems. FIG. 1 shows a computer system (also “system” herein) 101 programmed or otherwise configured to implement the methods of the disclosure, such as receiving sequencing data and classifying the presence or absence of genetic variants. The system 101 includes a central processing unit (CPU, also “processor” and “computer processor” herein) 105, which can be a single core or multi core processor, or a plurality of processors for parallel processing. The system 101 also includes memory 110 (e.g., random-access memory, read-only memory, flash memory), electronic storage unit 115 (e.g., hard disk), communications interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communications bus (solid lines), such as a motherboard. The storage unit 115 can be a data storage unit (or data repository) for storing data. The system 101 is operatively coupled to a computer network (“network”) 130 with the aid of the communications interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130 in some cases, with the aid of the system 101, can implement a peer-to-peer network, which may enable devices coupled to the system 101 to behave as a client or a server.

The system 101 is in communication with a processing system 140. The processing system 140 can be configured to implement the methods disclosed herein, such as mapping sequencing data to a reference sequence or assigning a classification to a genetic variant. The processing system 140 can be in communication with the system 101 through the network 130, or by direct (e.g., wired, wireless) connection. The processing system 140 can be configured for analysis, such as nucleic acid sequence analysis.

Methods and systems as described herein can be implemented by way of machine (or computer processor) executable code (or software) stored on an electronic storage location of the system 101, such as, for example, on the memory 110 or electronic storage unit 115. During use, the code can be executed by the processor 105. In some examples, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, can be compiled during runtime or can be interpreted during runtime. The code can be supplied in a programming language that can be selected to enable the code to execute in a pre-compiled, as-compiled or interpreted fashion.

Aspects of the systems and methods provided herein can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display that comprises a user interface (UI) for providing, for example, a customizable menu of genetic variants that can be analyzed by the methods of the disclosure. Examples of UI's include, without limitation, a graphical user interface (GUI) and web-based user interface.

In some embodiments, the system 101 includes a display to provide visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein. The display may provide one or more biomedical reports to an end-user as generated by the methods described herein.

In some embodiments, the system 101 includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

The system 101 can include or be operably coupled to one or more databases. The databases may comprise genomic, proteomic, pharmacogenomic, biomedical, and scientific databases. The databases may be publicly available databases. Alternatively, or additionally, the databases may comprise proprietary databases. The databases may be commercially available databases. The databases include, but are not limited to, MendelDB, PharmGKB, Varimed, Regulome, curated BreakSeq junctions, Online Mendelian Inheritance in Man (OMIM), Human Genome Mutation Database (HGMD), NCBI dbSNP, NCBI RefSeq, GENCODE, GO (gene ontology), and Kyoto Encyclopedia of Genes and Genomes (KEGG).

Data can be produced and/or transmitted in a geographic location that comprises the same country as the user of the data. Data can be, for example, produced and/or transmitted from a geographic location in one country and a user of the data can be present in a different country. In some cases, the data accessed by a system of the disclosure can be transmitted from one of a plurality of geographic locations to a user. Data can be transmitted back and forth among a plurality of geographic locations, for example, by a network, a secure network, an insecure network, an internet, or an intranet.

### User Interface

The system may comprise one or more user interfaces. The one or more user interfaces may be utilized to perform all or a portion of the methods disclosed herein. A user may select genetic variants to be queried prior to ordering the genetic test or the genetic variants may be selected after ordering the genetic test. A user of the methods can be, for example, a patient, a health-care provider, or a clinical laboratory (i.e., CLIA certified). In some cases, a first set of genetic variants may be selected for a first genetic test, and a second set of genetic variants may be later selected for a second genetic test. The second genetic test may comprise reanalyzing the sequencing data utilized for the first genetic test, analyzing new sequencing data, or analyzing a combination of both. The genetic variants selected for the second genetic test may be selected based on the analysis of the first genetic test. For example, a first clinically actionable variant identified in the first genetic test may indicate that the sequencing data should be analyzed for the presence or absence of a second clinically actionable variant. The healthcare provider or patient may select a panel of genetic variants for screening through a user interface. The panel of variants may be a plurality of variants grouped by disease type or subtype, phenotype, and the like. The panel of variants may comprise a plurality of clinically actionable variants known to be associated with a particular disease or phenotype. In some cases, the panel can be pre-set or pre-determined. Each set of variants can be customized and tailored to the patient's needs. For example, a user may select an entire pre-set panel of variants, may deselect one or more variants from the pre-set panel, or may add additional variants of interest to the pre-set panel. The additional variants may be variants that are associated with the disease or phenotype of the selected panel, or may be variants that are associated with a different disease or phenotype. A panel of variants may be updated based on scientific literature, genome studies, databases, and the like. For example, a variant may be added to the panel if the variant was previously classified as a variant of unknown significance (VUS) but has since been reclassified as a clinically actionable variant. Likewise, a variant may be removed from the panel if a clinically actionable variant is reclassified as benign.

The methods and systems as disclosed can utilize a pre-defined set of clinically actionable variants that can be assembled from one or more database, online source or published source. Non-limiting examples of published sources can include NCCN Clinical Practice Guidelines in Oncology, ESMO Oncology Clinical Practice Guidelines, AMP Clinical Practice Guidelines, and CAP IASLC AMP Molecular Testing Guidelines. Non-limiting examples of online sources can include the FDA Table of Pharmacogenomic Biomarkers in Drug Labeling (http://fda.gov/Drugs/ScienceResearch/ResearchAreas/Pharmacogenetics/ucm083378.htm) and the NCI Exceptional Responder Initiative database. Other non-limiting examples of databases can include MyCancerGenome (http://mycancergenome.com), PharmGKB (http://pharmgkb.org), MD Anderson Personalized Cancer Therapy Knowledge Base for Precision Oncology (http://pct.mdanderson.org). Other non-limiting examples of sources can include the clinical learning systems at major cancer centers, including IBM Watson and ASCO CancerLINQ. In some cases, the clinically actionable variant is a clinically actionable variant selected from Table 1.

### Performance

The methods and systems as disclosed herein can be utilized to improve the performance of identifying and/or classifying variants. The methods and systems disclosed herein can identify and/or classify genetic variants with a specificity of about or greater than about 50%, 55%, 60%, 65%, 70%, 75%, 76%, 77%, 78%, 79%, 80%, 85%, 86%, 87%, 88%, 89%, 90%, 91%, 92%, 93%, 94%, 95%, 96%, 97%, 98%, 99%, or 99.5. The methods and systems disclosed herein can identify and/or classify genetic variants with a sensitivity of about or greater than about 50%, 55%, 60%, 65%, 70%, 75%, 76%, 77%, 78%, 79%, 80%, 85%, 86%, 87%, 88%, 89%, 90%, 91%, 92%, 93%, 94%, 95%, 96%, 97%, 98%, 99%, or 99.5. The methods and systems disclosed herein can identify and/or classify genetic variants with a positive predictive value of about or at least about 80%, 85%, 90%, 95%, 95.5%, 96%, 96.5%, 97%, 97.5%, 98%, 98.5%, 99%, 99.5% or more. The methods and systems disclosed herein can identify and/or classify genetic variants with a negative predictive value of about or at least about 80%, 85%, 90%, 95%, 95.5%, 96%, 96.5%, 97%, 97.5%, 98%, 98.5%, 99%, 99.5% or more.

The methods and systems disclosed herein may increase the sensitivity when compared to the sensitivity of current methods. The methods and systems as described herein may increase the sensitivity by at least about 1%, 2%, 3%, 4%, 5%, 5.5%, 6%, 6.5%, 7%, 7.5%, 8%, 8.5%, 9%, 9.5%, 10%, 10.5%, 11%, 12%, 13%, 14%, 15%, 16%, 17%, 18%, 19%, 20%, 25%, 30%, 35%, 40%, 45%, 50%, 55%, 60%, 70%, 80%, 90%, 95%, 97% or more.

The methods and systems as described herein may increase the specificity by at least about 1%, 2%, 3%, 4%, 5%, 5.5%, 6%, 6.5%, 7%, 7.5%, 8%, 8.5%, 9%, 9.5%, 10%, 10.5%, 11%, 12%, 13%, 14%, 15%, 16%, 17%, 18%, 19%, 20%, 25%, 30%, 35%, 40%, 45%, 50%, 55%, 60%, 70%, 80%, 90%, 95%, 97% or more.

The methods and systems disclosed herein may identify variants with a mutation allelic fraction of at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 95%, 96%, 97%, 98%, 99% or more. In some cases, classifying has a sensitivity of at least 99%. In some cases classifying has a specificity of at least 99%. In some examples, each variant, when classified as present, has a mutant allele fraction of at least 5%. In other cases, each variant, when classified as present, has a mutant allele fraction of at least 10%. In some cases, classifying has a positive predictive value of at least 99%.

In some cases, the methods of the disclosure may be used to decrease the frequency of or eliminate false negatives (the inaccurately called “absence” of a genetic variant) in a sequencing data set as compared to alternative methods. The methods disclosed herein may decrease the frequency of false negatives as compared to alternative methods by about 1%, about 2%, about 3%, about 4%, about 5%, about 6%, about 7%, about 8%, about 9%, about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or about 100%. Additionally or alternatively, the methods of the disclosure may be used to decrease the frequency of or eliminate false positives in a sequencing data set as compared to alternative methods. The methods disclosed herein may decrease the frequency of false positives as compared to alternative methods by about 1%, about 2%, about 3%, about 4%, about 5%, about 6%, about 7%, about 8%, about 9%, about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or about 100%.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Identifying Genetic Variants in a Cohort of Cancer Samples

Sequencing will soon be an essential tool in the diagnostic workup of solid tumors. Of the more than 700 oncology drugs in the clinical development pipeline, 73% are expected to require a biomarker. Improved software systems are needed to manage the complexity of multiple-marker testing. A software system was built that would reliably deliver concordant results across variations in cancer type, tissue preservation, and target enrichment with high-performance, medical-grade analytics that could be readily validated and integrated into the solid tumor workflow at most pathology laboratories.

54 samples, from 5 different laboratories' published data, were chosen to represent a diverse mix of processing conditions and tumor types. The criterion for selection was the presence of one or more actionable variants in AKT, ALK, BRAF, BRCA1, CDKN2A, EGFR, KRAS, NRAS, PIK3CA, PIK3R1 or PTEN. 37 samples were from patient tumors, including lung, colon, esophageal and cancer of unknown primary, of which 18 were FFPE. 9 samples from circulating tumor cells (CTCs) were included, along with a dilution series of 8 cell line samples commonly used for laboratory validation. This study was performed using tumor-only data. The New Software System under evaluation was developed independently, configured with a pre-defined Test Panel of 156 variants, and then locked for the duration of the study. Identity-masked FASTQ files were processed as a single batch. The results were unmasked for comparison to the original published source.

The New Software System identified all actionable variants in 36 of 37 patient tumors, missing only 1 of 2 variants in a single sample. All of the cell line dilution series were correctly reported. 5 of the 9 samples were correctly reported in the CTC series, the remaining samples had 1 missed variant. With read depth below 30×, the missed calls in the CTC series point to inconsistent read depth as the cause for uneven performance in this specimen type. Across all patient tumor samples, successful calls had read depths of 50× to 2800×, suggesting a functional limit of detection of 50×. The New Software System demonstrated high concordance with cell line and patient solid tumor samples, both FFPE and frozen.

### Example 2. User Selection of Variant Panel

A user (i.e., healthcare practitioner or clinical laboratory) accesses a user portal of the disclosure. The user is presented with a menu of clinically actionable variants that can be selected for querying. The user can select a pre-set or pre-defined variant panel that comprises a plurality of clinically actionable variants related to a particular disease (e.g., prostate cancer). The user determines that two of the clinically actionable variants in the panel are not of interest and deselects or removes the two clinically actionable variants from the panel. The user also adds to the panel three genetic variants that have been recently described in a scientific publication as being correlated with treatment response in prostate cancer. The user saves the panel selection and transmits the panel selection to the server. The user uploads two FASTQ file formats to the server comprising target-enriched sequencing data of a patient suffering from prostate cancer. The computer processor identifies genomic regions of the sequencing data that contain the genetic addresses of the clinically actionable variants defined in the test panel. The computer processor identifies the presence or absence of each of the clinically actionable variants based on the methods of the disclosure. The computer processor generates a report listing the classification of each of the clinically actionable variants as well as treatment recommendations. The server transmits the report to the user portal for viewing by the user.

### Example 3. A New Software System Demonstrating High Concordance in Study with Multi-Laboratory Data

Sequencing will soon be an essential tool in the diagnostic workup of solid tumors. Of the more than 700 oncology drugs in the clinical development pipeline, 73% are expected to require a biomarker. Improved software systems are needed to manage the complexity of multiple-marker testing.

A new software system was constructed that would reliably deliver concordant results across variations in cancer type, tissue preservation, and target enrichment with high-performance, medical-grade analytics that could be readily validated and integrated into the solid tumor workflow at most pathology laboratories. Briefly described are findings from an initial verification study.

The goals of the study were to evaluate whether a single, standard analytic core can deliver consistent performance with data representing the broad range of conditions expected in clinical use: various tissue types and preservation; and multiple laboratories, protocols, and instruments; to evaluate whether our novel analytics, using tumor-only data, can provide equivalent results to more costly tumor-normal analytics; and to assess performance of the New Software System across a range of read depths. Common practice requires analytics “tuned” to a single laboratory protocol and instrument, so protocol changes can be highly disruptive. Further, common practice uses tumor-normal paired samples which may double the cost of testing.

Fifty-four (54) samples from five (5) different laboratories' published data were chosen to represent a diverse mix of processing conditions and tumor types as depicted in Table 2. The criterion for selection was the presence of one or more actionable variants in AKT, ALK, BRAF, BRCA1, CDKN2A, EGFR, KRAS, NRAS, PIK3CA, PIK3R1 or PTEN. This study was performed using tumor-only data as depicted in Table 3.

TABLE 2
Processing conditions at 5 laboratories

| Lab | Target Enrichment | Sequencer |
|---|---|---|
| Site 1 | SureSelect Custom | Illumina Genome Analyzer |
| | | IIx |
| Site 2 | SureSelect All Exon 50 MB | Illumina HiSeq 2000 |
| Site 3 | SureSelect Custom | Illumina HiSeq 2000 |
| Site 4 | Integrated DNA Technology, | Illumina HiSeq 2000 |
| | custom | |
| Site 5 | SureSelect All Exon v4 | Illumina HiSeq 2000 |

TABLE 3
Sample processing conditions

| Tumor Type | Preservation Method | # of Samples |
|---|---|---|
| NSCLC | FFPE | 3 |
| NSCLC CTC | Fresh | 9 |
| Colon | Fresh Frozen | 19 |
| Esophageal | FFPE | 10 |
| CUP | FFPE | 5 |
| LU Cancer Cell Line | Fresh | 8 |
| | | Total: 54 |

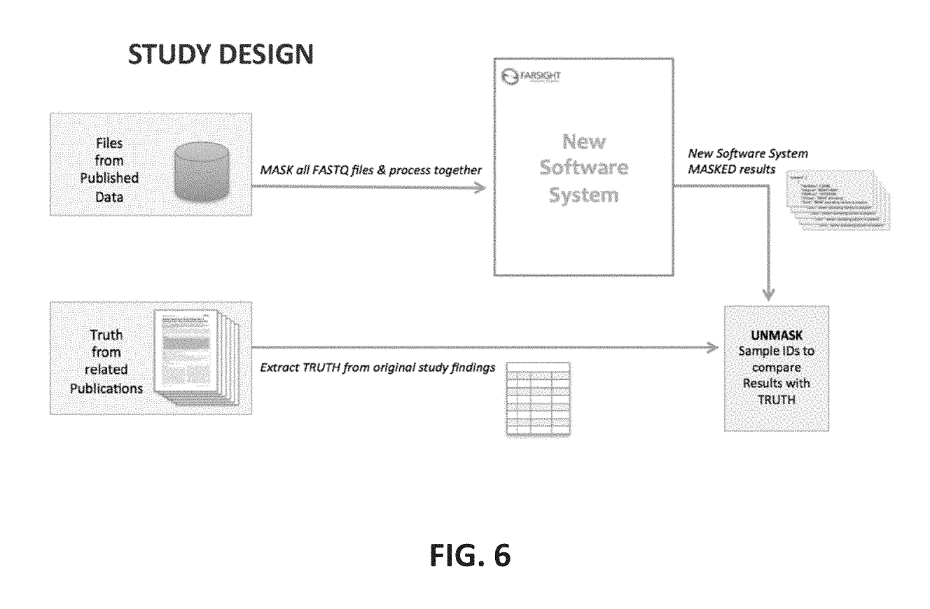

The New Software System under evaluation was developed independently, configured with a predefined Test Panel of 156 variants, and then locked for the duration of the study. Identity-masked FASTQ files were processed as a single batch. The results were unmasked for comparison to the original published source. FIG. 6 illustrates a workflow of the study design.

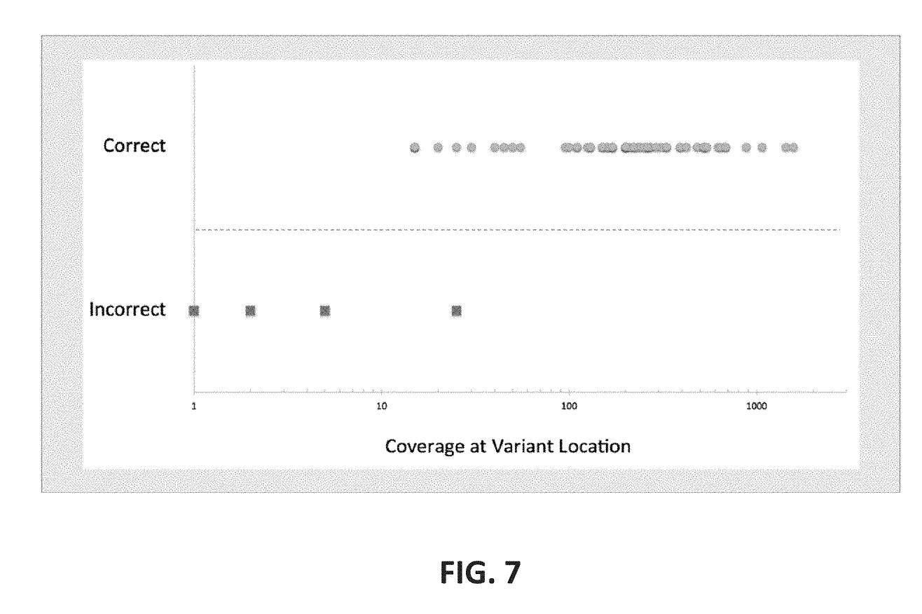

As depicted in Table 4 and FIG. 7, the New Software System identified all actionable variants in 36 of 37 patient tumors, missing only 1 of 2 variants in a single sample. All of the cell line dilution series were correctly reported. 5 of the 9 samples were correctly reported in the circulating tumor cell (CTC) series and the remaining samples had 1 missed variant. The 4 CTC samples with missed calls (Sample 46, Sample 49, Sample 51, and Sample 52), had read depths of <5×, <5×, 5× and 25×, respectively, at the putative variant location. These results establish a lower bound on the functional limit of detection. Read depths below 30× provide insufficient data to identify a variant at the designated location in these samples.

Sample 14 and Sample 31 were found to have amino acid substitutions in KRAS codon 12, which was misreported in the original publication. A detailed look at the reads in the KRAS codon 12 showed that Sample 14 carried a double mutation CC→AA, producing a G→F amino acid substitution. The results produced by the New Software System were verified using Integrative Genomics Viewer (IGV) and Ensembl Variant Effect Predictor (VEP).

TABLE 4
Results

| TRUTH as Published | | | | New Software System - Unmasked Results | | |
|---|---|---|---|---|---|---|
| Site 1 | Sample 1 | CO | BRAF.V600E | 22% | 330x | BRAF.V600E |
| Site 1 | Sample 2 | CO | BRAF.V600E | 34% | 200x | BRAF.V600E |
| Site 1 | Sample 3 | CO | BRAF.V600E | 28% | 130x | BRAF.V600E |
| Site 1 | Sample 4 | CO | KRAS.G12S, | 53%, 32% | 520x, 330x | KRAS.G12S, |
| | | | PIK3CA.E542K | | | PIK3CA.E542K |
| Site 1 | Sample 5 | CO | KRAS.G12C | 20% | 220x | KRAS.G12C |
| Site 1 | Sample 6 | CO | KRAS.G12D, | 20%, 24%, | 530x, | KRAS.G12D, |
| | | | PIK3R1.R358X, | 27% | 390x, 50x | PIK3R1.R358X, |
| | | | AKT.E17K | | | AKT.E17K |
| Site 1 | Sample 7 | CO | KRAS.G12C | 31% | 290x | KRAS.G12C |
| Site 1 | Sample 8 | CO | KRAS.G12D | 22% | 640x | KRAS.G12D |
| Site 1 | Sample 9 | CO | KRAS.G12V | 21% | 200x | KRAS.G12V |
| Site 1 | Sample 10 | CO | KRAS.G12D | 32% | 220x | KRAS.G12D |
| Site 1 | Sample 11 | CO | KRAS.G12A, | 27%, 57% | 170x, 150x | KRAS.G12A, |
| | | | BRCA1.N1067Y | | | BRCA1.N1067Y |
| Site 1 | Sample 12 | CO | KRAS.G12V, | 41%, 24% | 240x, 110x | KRAS.G12V, |
| | | | PIK3CA.E542K | | | PIK3CA.E542K |
| Site 1 | Sample 13 | CO | KRAS.A146T | 65% | 260x | KRAS.A146T |
| Site 1 | Sample 14 | CO | KRAS.G12N | 24% | 100x | KRAS.G12F* |
| Site 1 | Sample 15 | CO | KRAS.Q61H | 21% | 200x | KRAS.Q61H |
| Site 1 | Sample 16 | CO | NRAS.Q61K | 47% | 200x | NRAS.Q61K |
| Site 1 | Sample 17 | CO | NRAS.G12D | 25% | 250x | NRAS.G12D |
| Site 1 | Sample 18 | CO | PIK3CA.E545K | 27% | 420x | PIK3CA.E545K |
| Site 1 | Sample 19 | CO | none | n/a | n/a | none |
| Site 2 | Sample 20 | ESCC | PIK3CA.E542K | 52% | 125x | PIK3CA.E542K |
| Site 2 | Sample 21 | ESCC | PIK3CA.E545K | 40% | 270x | PIK3CA.E545K |
| Site 2 | Sample 22 | ESCC | PIK3CA.E545K | 14% | 160x | PIK3CA.E545K |
| Site 2 | Sample 23 | ESCC | PIK3CA.E545K | 23% | 110x | PIK3CA.E545K |
| Site 2 | Sample 24 | ESCC | PIK3CA.E545K | 42% | 170x | PIK3CA.E545K |
| Site 2 | Sample 25 | ESCC | PIK3CA.H1047R | 50% | 680x | PIK3CA.H1047R |
| Site 2 | Sample 26 | ESCC | PIK3CA.H1047R | 12% | 230x | PIK3CA.H1047R |
| Site 2 | Sample 27 | ESCC | PIK3CA.H1047L | 29% | 210x | PIK3CA.H1047L |
| Site 2 | Sample 28 | ESCC | CDKNA2.W110X | 25% | 25x | CDKNA2.W110X |
| Site 2 | Sample 29 | ESCC | none | n/a | n/a | none |
| Site 3 | Sample 30 | CUP | KRAS.G12C | 33% | 1570x | KRAS.G12C |
| Site 3 | Sample 31 | CUP | KRAS.G12C | 43% | 1070x | KRAS.G12A* |
| Site 3 | Sample 32 | CUP | PIK3CA.E545K | 31% | 1430x | PIK3CA.E545K |
| Site 3 | Sample 33 | CUP | CDKNA2.W110X | 32% | 170x | CDKNA2.W110X |
| Site 3 | Sample 34 | CUP | AKT.E17K | 49% | 260x | AKT.E17K |
| Site 4 | Sample 35 | LU Cancer | KRAS.G12S | 96% | 390x | KRAS.G12S |
| | | Cell Line | | | | |
| Site 4 | Sample 36 | LU Cancer | KRAS.G12C | 96% | 270x | KRAS.G12C |
| | | Cell Line | | | | |
| Site 4 | Sample 37 | LU Cancer | KRAS.G12C | 97% | 880x | KRAS.G12C |
| | | Cell Line | | | | |
| Site 4 | Sample 38 | LU Cancer | KRAS.G12C | 73% | 620x | KRAS.G12C |
| | | Cell Line | | | | |
| Site 4 | Sample 39 | LU Cancer | KRAS.G12C | 51% | 520x | KRAS.G12C |
| | | Cell Line | | | | |
| Site 4 | Sample 40 | LU Cancer | BRAF.G469A | 97% | 540x | BRAF.G469A |
| | | Cell Line | | | | |
| Site 4 | Sample 41 | LU Cancer | BRAF.G469A | 42% | 480x | BRAF.G469A |
| | | Cell Line | | | | |
| Site 4 | Sample 42 | LU Cancer | BRAF.G469A | 20% | 680x | BRAF.G469A |
| | | Cell Line | | | | |
| Site 5 | Sample 43 | NSCLC | EGFR.E746del | 37% | 310x | EGFR.E746del |
| Site 5 | Sample 44 | NSCLC | EGFR.E746del, | 93%, 51% | 160x, 95x | EGFR.E746del, |
| | | | PIK3CA.E545K | | | PIK3CA.E545K |
| Site 5 | Sample 45 | NSCLC | NRAS.Q61K | 46% | 150x | NRAS.Q61K |
| Site 5 | Sample 46 | NSCLC | EGFR.E746del, | 75% | <5x, 15x | EGFR.E746none, |
| | | CTC | PIK3CA.E545K | | | PIK3CA.E545K |
| Site 5 | Sample 47 | NSCLC | EGFR.E746del, | 100%, 85% | 40x, 55x | EGFR.E746del, |
| | | CTC | PIK3CA.E545K | | | PIK3CA.E545K |
| Site 5 | Sample 48 | NSCLC | EGFR.E746del, | 100%, | 20x, 15x | EGFR.E746del, |
| | | CTC | PIK3CA.E545K | 100% | | PIK3CA.E545K |
| Site 5 | Sample 49 | NSCLC | EGFR.E746del, | 81% | <5x, 15x | EGFR.E746none, |
| | | CTC | PIK3CA.E545K | | | PIK3CA.E545K |
| Site 5 | Sample 50 | NSCLC | NRAS.Q61K | 92% | 30x | NRAS.Q61K |
| | | CTC | | | | |
| Site 5 | Sample 51 | NSCLC | NRAS.Q61K | n/a | 5x | NRAS.none |
| | | CTC | | | | |
| Site 5 | Sample 52 | NSCLC | NRAS.Q61K | 15% | 25x | NRAS.Q61E |
| | | CTC | | | | |
| Site 5 | Sample 53 | NSCLC | NRAS.Q61K | n/a | 130x | NRAS.Q61K |
| | | CTC | | | | |
| Site 5 | Sample 54 | NSCLC | NRAS.Q61K | 11% | 45x | NRAS.Q61K |
| | | CTC | | | | |

*see explanation in description of results

The mismapping of variant to amino acid change, found in Sample 14 and Sample 31 is not uncommon in analytic pipelines designed for research use. These pipelines separate the variant calling from the effect prediction. In this way, effect prediction received insufficient information to recognize that two single nucleotide variants detected independently are present on the same reads, and thus share a codon with combined effect on the resultant amino acid.

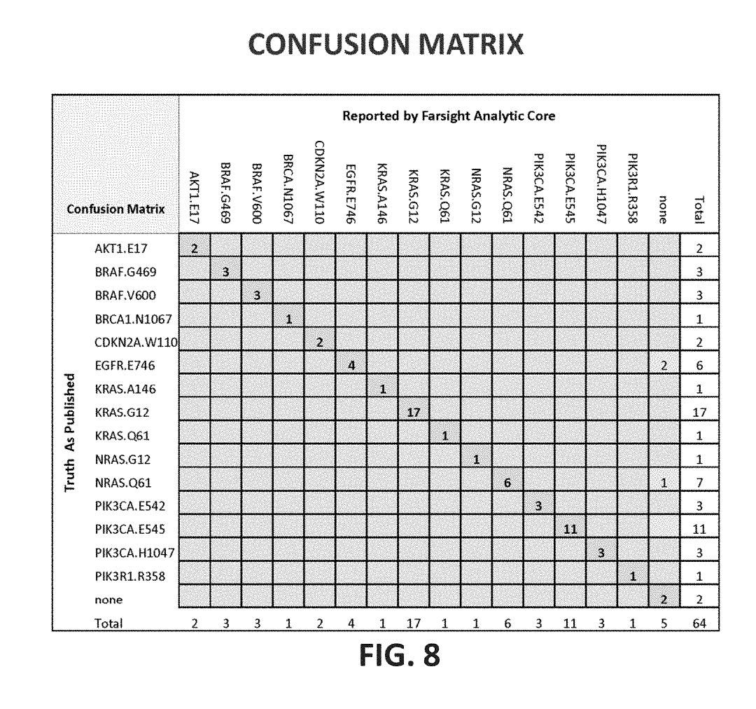

Every sample with read depth greater than 30× was called accurately by the New Software System, including those samples with challenging variants misreported by the original publications. FIG. 8 is a confusion matrix illustrating the performance of the algorithm.

In this initial verification study, the New Software System demonstrated high concordance with cell line and patient solid tumor samples, both formalin-fixed paraffin-embedded (FFPE) and frozen. The single, standard analytic core delivers consistent performance across the range of conditions expected in clinical use.

The algorithms in the New Software System enable tumor-only data to deliver results equivalent to more costly tumor-normal analytics. Accurate calls at read depths greater than 30× suggests that the generally accepted lower bound of 100× for clinical samples may be lowered when the New Software System is employed.

### Example 4. An Independent, Variant-Level Assessment Exposes Gaps in Probe Design and Coverage in Sequencing-Based EGFR Testing

EGFR inhibitors play an important role in the treatment of lung cancers with specific variants known to induce sensitivity or resistance to these targeted therapies. FDA-approved labels require testing for EGFR exon 19 deletions and exon 21 (L858R). The 2013 consensus guideline published by the Association for Medical Pathology (AMP), the College of American Pathologists (CAP) and the International Association for the Study of Lung Cancer (IASLC), and endorsed by the American Society of Clinical Oncology (ASCO), expanded this list to 26 EGFR variants, on exons 18, 19, 20, and 21, recommended for routine testing in lung adenocarcinomas.

Sequencing is often used in EGFR variant detection, but the method is sufficiently sensitive only if the processing protocol provides adequate coverage, or read depth, at the location where the variant is to be detected.

Whether the target enrichment protocols commonly used in sequencing-based testing provide consistent and adequate read depth at each of the Reportable Regions in the 2013 AMP/CAP/IASLC Guideline was assessed. To perform this assessment, a novel algorithm was built (CoverageFx), to perform a statistical assessment of read depth at each Reportable Region.

Data from 12 cohorts, sequenced by 11 different laboratories were chosen from published sources. Inclusion criteria were: 1) EGFR included in the target enrichment design; and 2) average read depth reported as 50× or greater.

The data included were generated using Illumina and Ion sequencers and target enrichment protocols from Agilent, Illumina, Ion and Raindance. Patient samples were from 10 different cancer types including lung, colon, breast, and melanoma. Each cohort was represented by 3-5 randomly chosen samples.

A total of 54 cancer patients samples sequenced at 11 different laboratories were obtained as FASTQ data files from publically available sources. These data were processed through the Farsight Analytic Core as described in Example 3. The results were grouped by cohort for post-processing using the CoverageFx algorithm to perform statistical assessment of read depth at each Reportable Region.

Table 5 summarizes processing characteristics that most influence read depth for each of the 12 cohorts included in the study. These include the target enrichment method, sequencer, tumor type and method of sample preservation. Each sequencing laboratory included an assessment of overall read depth as described in their respective original publications. The average local read depth for selected Reportable Regions is that computed by the CoverageFx algorithm. Across all EGFR Reportable Regions, the percent with average read depth below 100× is presented. For clinical use of sequencing data, a read depth of 100× is generally considered the minimum threshold at which a mutation present in 10% of tumor cells, in a biopsy containing as little as 20% tumor, can be detected.

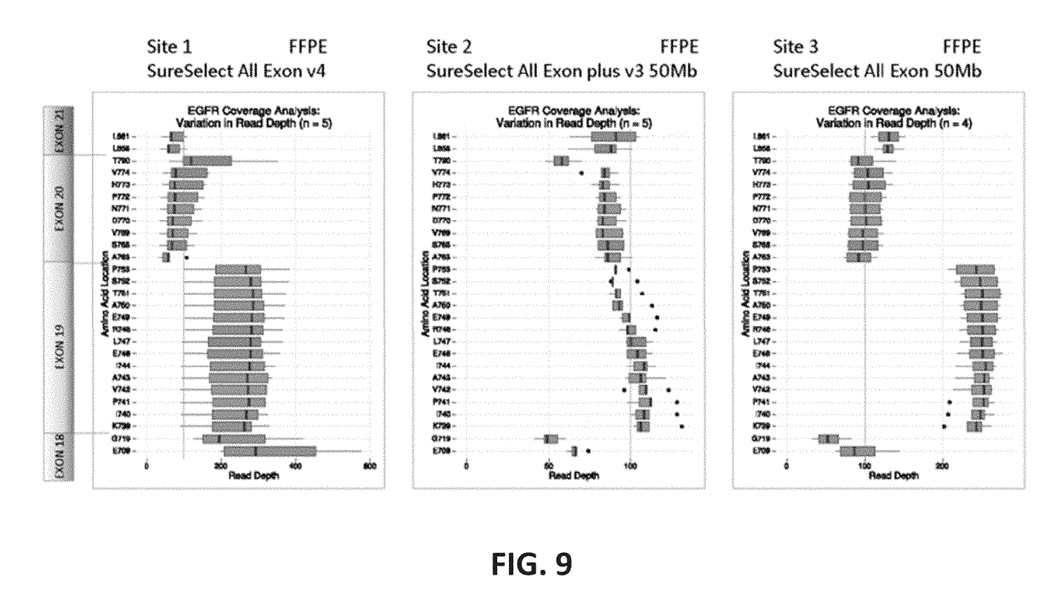

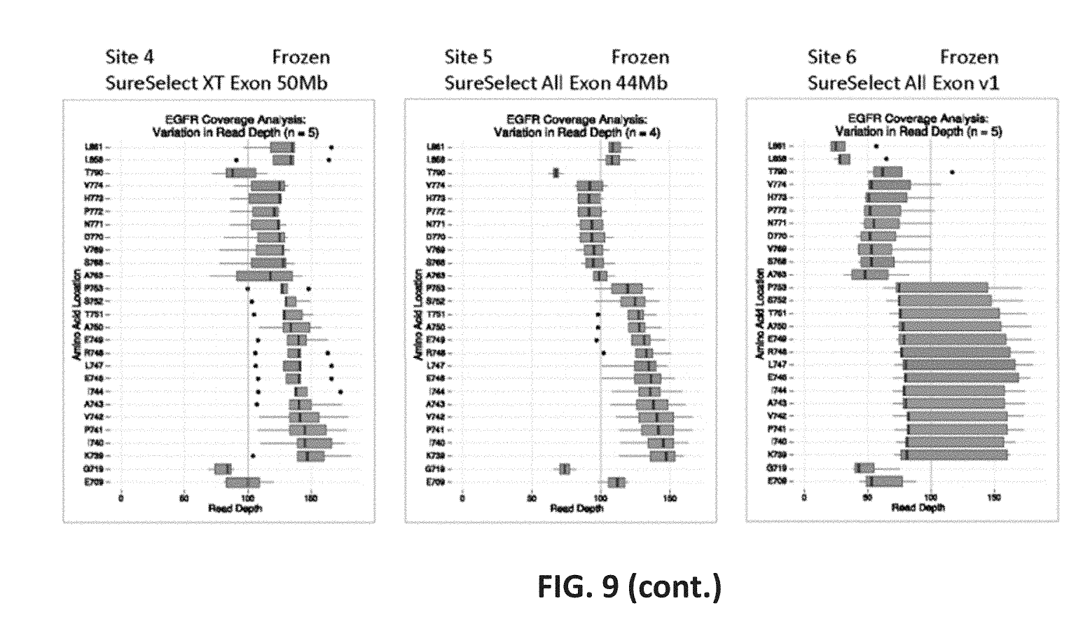

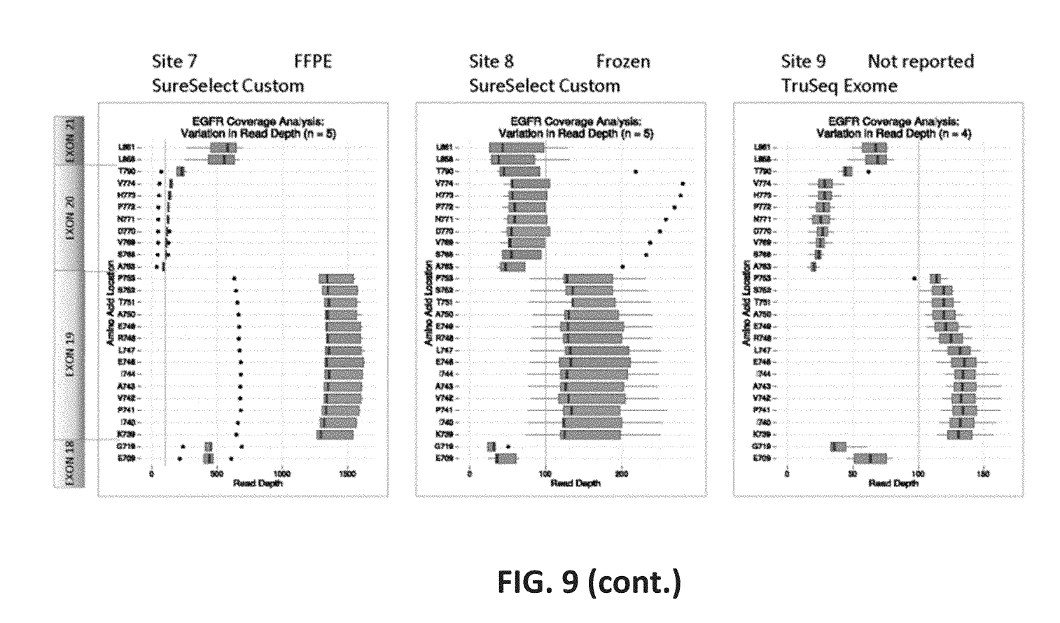

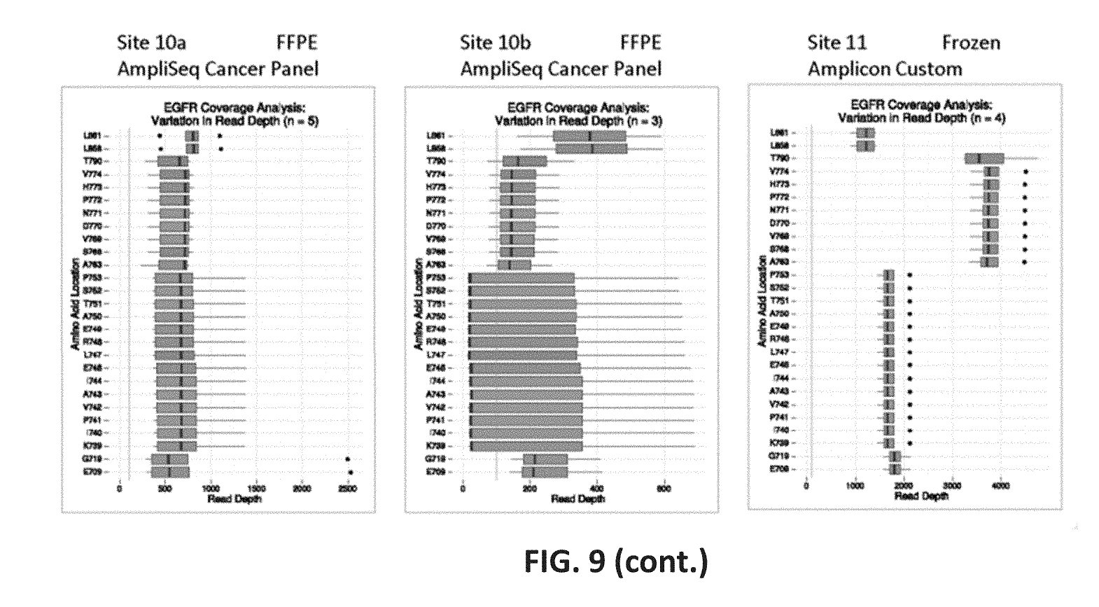

The statistical analysis performed by the CoverageFx algorithm was presented as box and whisker plots, shown for each cohort (FIG. 9).

The local read depth evaluated by CoverageFx, as shown in Table 5, exposes a large number of individual Reportable Regions with read depth below the clinical threshold of 100×. Although these cohorts may not have been sequenced with clinical intent, the differences are greater than one might expect given what was reported in the original publication. For a plurality of the cohorts analyzed, the resistance-causing T790 variant may have been missed due to below average read depths in that Reportable Region.

TABLE 5
Summary of cohorts included in the summary.

| | | | | | | | | | | % of |
|---|---|---|---|---|---|---|---|---|---|---|
| | | | | | | | | | | Reportable |
| | | | | | Overall | | | | | Regions |
| | | | | | Read | Average Local Read Depth at a | | | | with |
| | | | | | Depth | Reportable Region | | | | Average |
| | | | | | Reported | Exon | Exon | Exon | Exon | Read |
| | Target | | Tumor | Preservation | in Original | 18 | 19 | 20 | 21 | Depth |
| Site | Enrichment | Sequencer | Type | Method | Publication | G719 | E746 | T790 | L858 | <100x |
| Site 1 | SureSelect | Illumina | Lung | FFPE | 48-90x | 242x | 241x | 171x | 68x | 33% |
| | All Exon v4 | HiSeq | Adeno | | | | | | | |
| | | 2000 | | | | | | | | |
| Site 2 | SureSelect | Illumina | Bladder | FFPE | 79x | 50x | 104x | 58x | 84x | 63% |
| | All Exon | HiSeq | | | | | | | | |
| | Plus v3 | 2000 | | | | | | | | |
| | 50 Mb | | | | | | | | | |
| Site 3 | SureSelect | Illumina | Esophageal | FFPE | 79x | 54x | 249x | 100x | 130x | 19% |
| | All Exon | HiSeq | | | | | | | | |
| | 50 Mb | 2000 | | | | | | | | |
| Site 4 | SureSelect | Illumina | Lymphoma | Frozen | 129x | 80x | 137x | 92x | 129x | 11% |
| | XT Exon | HiSeq | | | | | | | | |
| | 50 Mb | 2000 | | | | | | | | |
| Site 5 | SureSelect | Illumina | Gastric | Frozen | 103x | 74x | 131x | 67x | 109x | 33% |
| | All Exon | HiSeq | | | | | | | | |
| | 44 Mb | 2000 | | | | | | | | |
| Site 6 | SureSelect | Illumina | Gastric | Frozen | 93-103x | 50x | 115x | 72x | 36x | 48% |
| | All Exon v1 | Genome | | | | | | | | |
| | | Analyzer | | | | | | | | |
| | | IIx | | | | | | | | |
| Site 7 | SureSelect | Illumina | CUP | FFPE | 458x | 450x | 1319x | 201x | 509x | 7% |
| | Custom | HiSeq | | | | | | | | |
| | | 2000 | | | | | | | | |
| Site 8 | SureSelect | Illumina | Colon | Frozen | 100x-435x | 32x | 157x | 68x | 61x | 30% |
| | Custom | Genome | | | | | | | | |
| | | Analyzer | | | | | | | | |
| | | IIx | | | | | | | | |
| Site 9 | TruSeq | Illumina | Lung | Not reported | 52x | 41x | 134x | 47x | 66x | 48% |
| | Exome | HiSeq | Adeno | | | | | | | |
| | | 2000 | | | | | | | | |
| Site | AmpliSeq | Ion | Melanoma, | FFPE | 290x-325x | 882x | 732x | 575x | 793x | 0% |
| 10a | Cancer | Torrent | Lung | | | | | | | |
| | Panel | PGM | Adeno | | | | | | | |
| Site | AmpliSeq | Ion | Colon | FFPE | 235x-315x | 255x | 238x | 189x | 383x | 0% |
| 10b | Cancer | Torrent | | | | | | | | |
| | Panel | PGM | | | | | | | | |
| Site | Amplicon | Illumina | Breast | Frozen | 1481x | 1826x | 1729x | 3771x | 1197x | 0% |
| 11 | Custom | MiSeq | | | | | | | | |

The broader statistical analysis performed by CoverageFx, as shown in the box and whisker plots for the 12 cohorts (FIG. 9), exposes otherwise hidden variation in read depth between Reportable Regions. For 8 of the 12 cohorts, differences are marked.

The EGFR exon 19 Reportable Region was consistently assessed at sufficient read depth across nearly all of the cohorts. This is not surprising, as exon 19 deletions are activating mutations that have been used for patient selection since early clinical trials, and are now on the labels of EGFR inhibitors. By contrast, exons 18, 20 and 21 were all under-sampled in key regions. The important Reportable Region in exon 20, T790, was measured at sufficient read depth in just 50% of the cohorts. On exon 21, the important L858 region, as well as exon 18 Reportable Regions were measured at sufficient read depth in only 42-58% of the cohorts. Important differences in target enrichment emerge, with marked improvement in read depth in exons 18, 20 and 21 of more recent versions of all exon target enrichment products.

This multi-cohort study demonstrates that average coverage alone is an inadequate, even misleading, quality measure in clinical sequencing. The CoverageFx algorithm used in this study exposed significant, unexpected variation in coverage across key Reportable Regions.

This study underscores the importance for laboratories performing sequencing-based testing to confirm read depth sufficiency at each reportable region. Such read depth confirmation should be minimally performed at the time of test validation. Ideally, read depth should be confirmed for each Reportable Region with each patient report.

### Example 5. Indication-Specific Reporting

A sequencing data input is received by the system of the disclosure. The sequencing data input can be from a sequencer (e.g., Illumina sequencer) or from a data repository. The system identifies the presence or absence of clinically actionable variants related to three different indications. Choosing indications that have a significant gene list overlap optimizes the cost of operating the system. A user (i.e., healthcare practitioner or clinical laboratory) accesses a user portal of the disclosure. The user has the option of selecting from three reports. Each of the three reports provides information related to the presence or absence of clinically actionable variants for a respective indication. The computer processor generates a report listing the classification of each of the clinically actionable variants as well as treatment recommendations. The server transmits the report to the user portal for viewing by the user.

### Example 6. Dual Output System

A user (i.e., healthcare practitioner or clinical laboratory) accesses a user portal of the disclosure. The user is presented with a menu of clinically actionable variants that can be selected for querying. The user can select a pre-set or pre-defined variant panel that comprises a plurality of clinically actionable variants related to a particular disease (e.g., prostate cancer). The user determines that two of the clinically actionable variants in the panel are not of interest and deselects or removes the two clinically actionable variants from the panel. The user also adds to the panel three genetic variants that have been recently described in a scientific publication as being correlated with treatment response in prostate cancer. The user further selects a plurality of genes/variants that are requested by a clinical trial sponsor. The user saves the panel selection and transmits the panel selection to the server. The user uploads two FASTQ file formats to the server comprising target-enriched sequencing data of a patient suffering from prostate cancer. The user optionally uploads a clinical trial eligibility report to the system which contains information related to the patient (e.g., biographical data, health risk assessment, etc). The computer processor identifies genomic regions of the sequencing data that contain the genetic addresses of the clinically actionable variants defined in the test panel. The computer processor identifies the presence or absence of each of the clinically actionable variants based on the methods of the disclosure. The computer processor generates a report listing the classification of each of the clinically actionable variants as well as treatment recommendations. The computer processor generates a separate report listing the classification of the additional genes/variants requested by the clinical trial sponsor. The server transmits the combined report to the user portal for viewing by the user. The user can share access to the user portal with the clinical trial sponsor or can relay the report to the clinical trial sponsor.

### Example 7. Parallel Analysis System

A user (i.e., healthcare practitioner or clinical laboratory) accesses a user portal of the disclosure. The user is presented with a menu of clinically actionable variants that can be selected for querying. The user can select a pre-set or pre-defined variant panel that comprises a plurality of clinically actionable variants related to a particular disease (e.g., prostate cancer). The user determines that two of the clinically actionable variants in the panel are not of interest and deselects or removes the two clinically actionable variants from the panel. The user also adds to the panel three genetic variants that have been recently described in a scientific publication as being correlated with treatment response in prostate cancer. The user saves the panel selection and transmits the panel selection to the server. The user uploads two FASTQ file formats to the server comprising target-enriched sequencing data of a patient suffering from prostate cancer. The computer processor identifies genomic regions of the sequencing data that contain the genetic addresses of the clinically actionable variants defined in the test panel. The computer processor identifies the presence or absence of each of the clinically actionable variants based on the methods of the disclosure. The system further utilizes a multi-marker algorithm designed by a third party. The computer processor generates a report listing the classification of each of the clinically actionable variants as well as treatment recommendations. The computer processor integrates computations using the multi-marker algorithm into the report. The server transmits both reports to the user portal for viewing by the user.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for detecting the presence or absence of a genetic variant, comprising:
(a) receiving a data input comprising sequencing data generated from a nucleic acid sample from a subject;
(b) determining a presence or absence of said genetic variant from said sequencing data, wherein said determining comprises assigning a quality score to a genomic region comprising said genetic variant, wherein said assigning is performed by a computer processor;
(c) classifying said genetic variant based on said quality score to generate a classified genetic variant, and
(d) outputting a result based on said classifying, thereby identifying said classified genetic variant,
wherein said classifying further comprises classifying said genetic variant as present if said genetic variant is determined to be present and said quality score for said genomic region comprising said genetic variant is greater than a predetermined threshold,
wherein said classifying further comprises classifying said genetic variant as absent if said genetic variant is determined to be absent and said quality score for said genomic region comprising said genetic variant is greater than a predetermined threshold, and
wherein said classifying further comprises classifying said genetic variant as indeterminate if said quality score for said genomic region comprising said genetic variant is less than a predetermined threshold.

2. The method of claim 1, wherein said outputting a result comprises generating a report, wherein said report identifies said classified genetic variant.

3. The method of claim 1, further comprising mapping said sequencing data to a reference sequence.

4. (canceled)

5. (canceled)

6. The method of claim 1, wherein said predetermined threshold comprises a depth of coverage of said genomic region comprising said genetic variant.

7. The method of claim 6, wherein said depth of coverage is at least 10×.

8-11. (canceled)

12. The method of claim 1, wherein said predetermined threshold comprises a confidence score.

13. The method of claim 12, wherein said confidence score is at least 95%.

14. (canceled)

15. The method of claim 1, wherein said genetic variant comprises a clinically actionable variant.

16. The method of claim 15, wherein said identifying said classified genetic variant further indicates a treatment for said subject based on said classified genetic variant.

17. (canceled)

18. (canceled)

19. The method of claim 16, wherein said subject is administered a treatment based on said result.

20. The method of claim 15, wherein said clinically actionable variant is in a gene that alters a response of said subject to a therapy.

21. (canceled)

22. The method of claim 15, wherein a presence of a clinically actionable variant indicates said subject is a candidate for a specific therapy.

23. The method of claim 15, wherein an absence of a clinically actionable variant indicates said subject is not a candidate for a specific therapy.

24-31. (canceled)

32. The method of claim 1, wherein said genetic variant is a gene amplification, an insertion, a deletion, a translocation or a single nucleotide polymorphism.

33. The method of claim 1, wherein said sequencing data comprises target-enriched sequencing data.

34. The method of claim 33, wherein said target-enriched sequencing data comprises whole exome sequencing data.

35. The method of claim 1, wherein said sequencing data comprises whole genome sequencing data.

36. The method of claim 1, wherein said classifying has a sensitivity of at least 99%.

37. The method of claim 1, wherein said classifying has a specificity of at least 99%.

38. The method of claim 1, wherein said genetic variant, when classified as present, has a mutant allele fraction of at least 5%.

39. (canceled)

40. The method of claim 1, wherein said classifying has a positive predictive value of at least 99%.

41. The method of claim 1, wherein said quality score is based on at least one of a depth of coverage, a mapping quality, or a base call quality.

42-44. (canceled)

45. The method of claim 1, further comprising, prior to step (a), sequencing said nucleic acid sample from said subject to generate said sequencing data.

46. The method of claim 1, further comprising requerying said sequencing data to determine a presence or an absence of one or more additional genetic variants, comprising assigning a quality score to each of one or more genomic regions comprising said one or more additional genetic variants, wherein said quality score is classified as sufficient if said quality score is greater than a predetermined threshold and wherein said quality score is classified as insufficient if said quality score is lower than a predetermined threshold.

47. The method of claim 1, wherein said quality score is determined by a total read depth at a specific location of said genetic variant, a proportion of reads containing said genetic variant, the mean quality of non-variant base calls at said location of said genetic variant, and the difference in mean quality for variant base calls.

48. The method of claim 47, wherein said quality score is determined by a machine learning algorithm.

49-131. (canceled)
